(19) United States
(12) Patent Application Publication
(10) Pub. No.: US 2005/0238616 A1
(43) Pub. Date: Oct. 27, 2005

(54) Compositions for treatment of central nervous system neoplasms, and methods of making and using the same

(75) Inventors: Wenbin Dang, Belle Meade, NJ (US); Dana Hilt, Ellicott City, MD (US); William Vincek, Baltimore, MD (US)

Correspondence Address: FOLEY HOAG LLP;PATENT GROUP, WORLD TRADE CENTER WEST, 155 SEAPORT BOULEVARD, BOSTON, MA, 02110-2600, US

(73) Assignee: Guilford Pharmaceuticals, Inc., Baltimore, MD (US)

(21) Appl. No.: 10/500,795

(22) PCT Filed: Jan. 9, 2003
(86) PCT No.: PCT/US03/00564

§ 371 Date: Dec. 3, 2004

Related U.S. Application Data
(60) Provisional application No. 60/347,081, filed on Jan. 9, 2002.

(51) Int. Cl. A61K 31/74

(52) U.S. Cl. 424/78.17

(57) ABSTRACT

The present invension relates to compositions of a biocompatible polymer and a antineoplastic agent for treating central nervous system neoplasms, and methods of using and making the same. In certain embodiments, the polymer contains phosphorous linkages. In other embodiments, the antineoplastic agent is an antineoplastic taxane. In still other embodiments, the subject methods of treatment use electromagnetic radiation.

### INTRODUCTION

The blood-brain barrier (including the blood-cerebrospinal fluid barrier) regulates the movement of substances between the circulatory system and the central nervous system (CNS). The capillaries that supply the blood to the brain and the spinal column have tight junctions which block passage of most molecules through the capillary endothelial membranes. While the membranes often allow passage of lipid soluble materials, water soluble materials such as glucose, proteins and amino acids do not typically diffuse passively through the barrier. Mediated transport mechanisms assist the movement of glucose and essential amino acids across the blood-brain barrier. Mediated transport mechanisms also remove molecules which become in excess, such as potassium, from the brain.

The blood-brain barrier impedes the delivery of drugs to the CNS. Pharmaceuticals that are available to treat disorders in other parts of the body are often difficult to utilize in the treatment of conditions that occur within the confines of the blood-brain barrier. A variety of methods have been devised for the delivery of pharmaceuticals to the CNS. One common approach is to modify the hydrophobicity of a compound in an effort to produce a form that crosses the blood-brain barrier. Another approach is to use a prodrug that crosses the barrier and is then metabolized within the CNS to produce the active drug form. Such chemistry-based methods are limited to pharmaceuticals that can be appropriately modified and still meet safety and efficacy requirements. In an alternative approach, the pharmaceutical may be introduced directly into the CNS through injection, implantation or other means. In some cases this approach has been successful, however the invasiveness of the procedure may preclude repeated dosing. In addition, biocompatibility between the pharmaceutical composition and the CNS is sometimes problematic. Despite the absence of many components of the immune system in the CNS, transplanted substances often cause a foreign body or other inflammatory response. The CNS is relatively intolerant of volume changes and cellular damage that can accompany such responses, and therefore substances to be introduced directly into the CNS may cause as little inflammation as possible. Accordingly, it would be beneficial to have improved methods for the introduction of pharmaceuticals behind the blood-brain barrier.

Many diseases affect the CNS, most of which might be treated more effectively with an improved method for introduction to the CNS. One type of CNS disease is neoplasm. CNS neoplasms involve a large array of anatomical sites in the cranium, brain and spinal cord. This type of neoplasm is difficult to treat, is often life-threatening, and severely compromises patient quality of life. Many patients, as a result of the disease and its treatment, must endure alterations in cranial, facial, and neck appearance as well as alterations in cognition, memory, motor skills, coordination, mood, speech, sight, smell, chewing, swallowing, touch and taste perception. Treatment of CNS neoplasm is complicated and may vary from person to person, often depending on the size and location of the tumor. Typically a physician will select a treatment regimen that minimizes disfigurement yet is most likely to be effective in reducing tumor growth.

The most frequently occurring type of CNS neoplasm is glioma, arising from any of the stromal (supporting) cell types of the CNS. Gliomas include astrocytomas, glioblastoma multiforme, medulloblastomas, oligodendrogliomas, and ependymomas. Any of these gliomas may occur in a variety of grades that reflect aggressiveness of the neoplasm, degree of malignancy, and predisposition for metastasis. Neoplasms of neurological origin account for approximately ten percent of all neoplasms and are the most common solid tumors of childhood. Incidence is roughly equal between the sexes for most tumor types.

Other types of CNS tumors include meningiomas, pituitary tumors, neurilemomas, metastatic tumors from CNS and other tissue types, endotheliomas and other blood vessel tumors, dermoids, epidermoids, teratomas, chordomas, paraphyseal cysts, craniopharyngiomas, germinomas or pinealomas, and other tumor types, including sarcomas, papillomas of the choroid plexus, lipomas, lymphoma, and unclassified tumors.

CNS tumors typically occur within the bony confines of the cranium and vertebral column and thus may cause generalized disease by exerting pressure on nearby and even distant regions of the CNS. Local disease caused by a tumor may result from irritation of adjacent brain tissue, destruction of adjacent tissue by invasion or by secretion of cytotoxic materials, or compression of nearby structures due to tumor growth. Neoplasms may also predispose an organism to hemorrhage from the fragile blood vessels that may form inside tumors.

CNS cancers share many traits in common with other solid tumors, including local growth, and distant metastasis, both within the CNS and in other organs. Most significant for these tumors, however, is their anatomic location and the consequent effect of local growth on disruption of essential functions. The tissues of the CNS play a central role in permitting the human being to eat, speak and breathe normally. Normal function of these tissues is also highly significant to self-image, body image and social interaction. In the CNS, vital structures for these functions, including various anatomical regions of the brain and spinal cord, as well as skin, muscle, nerve, bone, and blood vessels, are all in close proximity. The impact of CNS tumors on the patient is related to their ability to disrupt normal functions in the areas where they originate and into which they spread. The spread of an invasive cancer into the vital structures of the CNS may cause major, potentially lethal, disruption of vital human functions. A large proportion of patients who die from CNS neoplasms have no clinical evidence of tumor spread beyond this anatomic region. Instead, local phenomena such as cerebrospinal fluid obstruction, local infection, rupture of a major vessel, CNS metastasis, and other systemic diseases are responsible for the deaths.

Because these tumors predominately cause their morbidity and mortality by local and locoregional spread, successful treatment of the disease in the CNS may be necessary for extending lifespan and maintaining quality of life.

A variety of treatment regiments are known. Basic principles of solid tumor therapy may be applied to CNS neoplasms. As with most solid tumors, surgical treatment or radiation is carried out to provide en bloc extirpation of the primary tumor and the regional metastases. As with other solid tumors, protocols have been established for CNS neoplasms to evaluate the usefulness of adjuvant and neoadjuvant systematic chemotherapy when combined with locoregional control. Alternatively, chemotherapy alone may be used.

In all these cases, high recurrence rates after treatment is a significant problem. Approximately half of patients with gliomas have advanced stage disease, either grade 3 or stage 4. Such advanced disease has a high recurrence rate even after aggressive initial therapy. Even when initial disease is less advanced, local recurrence may occur. Recurrent disease may involve the primary tumor site, or the contralateral side. At times, resection of the area of recurrence is possible. Recurrent disease is responsible for some of the most devastating and difficult to treat sequelae of CNS tumor. Recurrent tumors may cause pain, destruction of function, or invasion of vital structures such as nerves, blood vessels or cognition or sensation centers in the brain. Recurrence may also indicate that the cancer has become incurable, with resultant prolonged and poorly treatable deterioration of quality of life.

In part, there remains a need in the art for systems to deliver drugs to treat CNS neoplasms.

### SUMMARY OF THE INVENTION

It is an object of the invention to provide compositions and methods for introducing substances into the CNS. In general such substances will be incorporated with a polymer that provides sustained release of the substance in vivo. In many embodiments, the substance will have therapeutic effects on a disease or condition affecting the CNS. It is further understood that such substances may be administered as a sole treatment or in combination with surgical and/or other interventions, such as, for example, pharmacological treatments.

In certain embodiments, the present invention provides methods and compositions for the treatment of CNS neoplasms. It is one object of the present invention to supplement primary extirpative procedures performed to treat cancers of the CNS, for example, gliomas and gliosarcomas. It is a further object to provide control of microscopic residual disease after surgical excision of a CNS cancer. In other embodiments, the present invention may be useful in treating or supplementing the treatment of a local excisional site for other types of cancers found in the CNS area, for example, meningiomas and metastatic tumors. As will be understood by practitioners skilled in the art, the compounds and methods of the present invention may be effective in the treatment of a variety of tumor types, including those tumors known to be or discovered to be responsive to an antineoplastic agent.

In one embodiment, the present invention may be directed to enhancing local control of excised tumors that have or are likely to have positive margins. In other embodiments, the present invention may be directed to enhancing local control of excised tumors that have a significant likelihood of local recurrence. In certain cases, the availability of the present invention as a supplement to a cranial dissection may permit the surgeon to select a less extensive, functional or structure-preserving technique for cranial dissection instead of a more extensive or radical cranial dissection. In certain embodiments, the present invention may be particularly advantageous in surgeries where there is a significant likelihood of recurrent disease in the CNS.

It is a further object of the present invention to be injectable, implantable, infusible or otherwise directable into an afflicted area of the CNS, whether a primary or a recurrent tumor, an area of stroke damage, an area affected by Parkinson's disease, etc. The present invention may be prepared to include an appropriate vehicle for this injection, implantation, infusion or direction. In other embodiments, the present invention may be prepared for intravascular infusion or for topical administration into an anatomic region of the CNS where treatment is desired. In certain embodiments, intravascular infusion may involve an intra-arterial injection into a native artery feeding the CNS, or may involve an intra-arterial injection into an artery feeding a transposed or transplanted reconstructive tissue associated with the CNS.

In certain aspect, the present invention may be used in conjunction with immediate or delayed reconstructive procedures in order to decrease the likelihood of recurrent disease or to increase the adequacy of tumor extirpation or local control related to the CNS in circumstances where reconstruction is being performed. In some embodiments, compositions of the present invention may be positioned in a surgically created defect of the CNS that is to be reconstructed, and is to be left in this position after the reconstruction has been carried out. The present invention may be suitable for use with local tissue reconstructions of the CNS.

It is an object of the present invention to be used in combination with other treatment modalities in certain embodiments. As examples, the systems and methods of the present invention may be used in conjunction with surgery, with radiation, with systemic chemotherapy or with a combination of these modalities.

In certain embodiments, electromagnetic radiations may be used to treat a CNS neoplasm in conjunction with the subject compositions. The radiation treatment may be completed before, after, or concomitant with administration of a subject composition. As described in greater detail below, the order of radiation treatment may affect the results of any such therapies.

According to certain embodiments of the present invention, these objects and other desirable results may be accomplished in treating a CNS neoplasm by administering a therapeutically effective amount of a composition comprising a biocompatible and optionally biodegradable polymer and an antineoplastic agent suitable for such disease. In certain practices of the present invention, the anatomic area of the CNS being treated may be reached by an access device that conveys, transports, instills or delivers the composition of the present invention to the preselected anatomic location. In part, the present invention is directed to a polymer system for use in the above-described treatments, such as a biocompatible polymer, comprising an antineoplastic taxane (e.g., paclitaxel), methods for treatment using the subject compositions, and methods of making and using the same.

In certain embodiments, a large percentage of the subject compositions may be an antineoplastic agent, such as an antineoplastic taxane, that may be used to treat a CNS neoplasm. For example, such an agent may comprise 5% to 60% or more of the subject composition, such as at least about 10%, at least about 30%, or at least about 50% of said agent.

In certain embodiments, administration of the subject polymers results in sustained release of an encapsulated antineoplastic agent for an extended period of time and in an amount that is not possible with other modes of administration. In certain embodiments, release of the antineoplastic agent follows zero order kinetics, i.e. the rate of release is independent of the concentration of antineoplastic agent present. In some instances there will be an initial burst, or higher rate of release, followed by a steady zero-order release. In certain embodiments, the properties of the polymer:therapeutic complex are such that the burst is minimized.

The subject compositions, and methods of making and using the same, achieve a number of desirable results and features, one or more of which (if any) may be present in any particular embodiment of the present invention: (i) a single dose of a subject composition may achieve the desired therapeutically beneficial response to treat a CNS neoplasm through sustained release of an antineoplastic agent; (ii) sustained release of an antineoplastic agent from a biocompatible and optionally biodegradable polymer composition in an anatomic area of the CNS; (iii) novel treatment regimens for treating primary, recurrent or locally metastatic CNS neoplasms using the subject compositions for sustained delivery of an antineoplastic agent; (iv) high levels of loading (by weight), e.g. greater than 10% and up to 60% or more, of an antineoplastic agent for treatment of a CNS neoplasm in biocompatible polymers; and (v) lyophilization or subjection to an appropriate drying technique such as spray drying of the subject compositions and subsequent rehydration.

In one aspect, the subject polymers may be biocompatible, biodegradable or both. In certain embodiments, the subject polymers contain phosphorus linkages, including, for example, phosphate, phosphonate and phosphite. In other embodiments, the monomeric units of the present invention have the structures described in the claims appended below, which are hereby incorporated by reference in their entirety into this Summary. In the subject polymers, and in particular in those embodiments containing a phosphorus linkage, the chemical structure of certain of the monomeric units may be varied to achieve a variety of desirable physical or chemical characteristics, including for example, release profiles or handling characteristics of the resulting polymer composition.

In certain embodiments, other materials may be encapsulated in the subject polymer in addition to the antineoplastic agent used to treat a CNS neoplasm to alter the physical and chemical properties of the resulting polymer, including for example, the release profile of the resulting polymer composition for such agent. Examples of such materials include biocompatible plasticizers, delivery agents, fillers and the like.

The present invention provides a number of methods of making the subject compositions. Examples of such methods include those described in the Exemplification below.

In certain embodiments, the subject compositions are in the form of microspheres. In other embodiments, the subject compositions are in the form of nanospheres. In one aspect, the subject compositions of the present invention may be lyophilized or subjected to another appropriate drying technique such as spray drying and subsequently rehydrated for ready use.

In another aspect, the present invention is directed to methods of using the subject polymer compositions for prophylactic or therapeutic treatment of CNS neoplasms and their related conditions and symptoms. In certain instances, the subject compositions may be used to prevent such a disease or condition. In certain embodiments, use of certain of the subject compositions, which release in a sustained manner an antineoplastic agent, allow for different treatment regimens for CNS neoplasms than are possible with other modes of administration of such an antineoplastic agent.

In another aspect of the invention, the efficacy of treatment using the subject compositions (optionally with electromagnetic radiation) may be compared to treatment regimens known in the art in which an antineoplastic agent is not encapsulated within a subject polymer or other treatment regimens. For example, treatment with a subject composition is expected to result in fewer hypersensitivity reactions than treatment with an antineoplastic agent such as paclitaxel, with or without premedication. Alternatively, treatment with a subject composition results in an increase in the median survival rate in mice, and it is expected that the same will result in other mammals, and in particular humans. Alternatively, the efficacy of treatment with a subject composition may be greater than with treatment with an antineoplastic agent alone or in a pharmaceutically acceptable carrier.

In another aspect, the subject polymers may be used in the manufacture of a medicament for any number of uses, including for example treating any disease or other treatable condition of a patient. In still other aspects, the present invention is directed to a method for formulating polymers of the present invention in a pharmaceutically acceptable carrier.

In another aspect, the present invention may be spray dried and subsequently rehydrated for ready use or injected as powder using appropriate powder injecting device.

In other embodiments, this invention contemplates a kit including subject compositions, and optionally instructions for their use. Uses for such kits include, for example, therapeutic applications. In certain embodiments, the subject compositions contained in any kit have been lyophilized and require rehydration before use.

The embodiments and practices of the present invention, other embodiments, and their features and characteristics, will be apparent from the description, drawings and claims that follow.

### BRIEF DESCRIPTION OF THE FIGURES

FIG. 1 illustrates in vitro release of paclitaxel from PACLIMER (10%) (which is described in more detail below). Release was measured from 10 mg of PACLIMER (10%) in PBS at 37° C. Release was measured from free microspheres (□) and microspheres compressed into discs with 0% (◯), 50% (*), and 75% (∇) PEG-1000 as an excipient. Results are expressed as cumulative total release as a percentage of initial loading over time.

FIG. 2 illustrates in vivo biodistribution of paclitaxel delivered from PACLIMER (10%) microspheres in the rat brain. ³H paclitaxel was incorporated into PACLIMER (10%) loading and compressed into 10 mg discs containing 50% PEG-1000 by weight for implantation in the rat parietal lobe. Brains were removed at 7- and 30-days post-implantation and divided into (A) implant and (B) contralateral hemispheres. Each hemisphere was sectioned at 2-mm intervals coronally and the amount of paclitaxel present in the section was quantitated by liquid scintillation counting. Measurements represent the mean of three separate animals +/−SEM.

FIG. 3 shows Kaplan-Meier survival curves of rats bearing established intracranial 9L gliosarcoma tumors treated with 10 mg PACLIMER (10%):PEG-1000 (50:50) implants. (A) Systemically, administered PACLIMER (10%) did not prolong survival compared to animals receiving no treatment (median PACLIMER (10%)=14d, median control=18d, p=0.0944). (B) PACLIMER [symbol] , implanted intratumorally extended median survival compared to animals receiving no treatment (PACLIMER (10%)—33d, control-20d. p<0.0001) or the polymer used in PACLIMER:PEG-1000 microspheres with no paclitaxel (experiment 2, PACLIMER=33d, polymer used in PACLIMER=17d,p<0−0001). (C) PACLIMER extended median survival compared to control groups receiving the polymer used in PACLIMER:PEG-1000 microspheres with no paclitaxel (PACLIMER=35d, the polymer used in PACLIMER=16d, p<0.001).

FIG. 4 shows Kaplan-Meier survival curves for the experiment described in Example 13E below, which uses electromagnetic radiation in combination with PACLIMER (10%).

### DETAILED DESCRIPTION OF THE INVENTION

1. Overview

The present invention relates to pharmaceutical compositions for the delivery of antineoplastic agents, including antineoplastic taxanes such as paclitaxel, for the treatment of CNS neoplasms. CNS neoplasms include a large variety of disorders affecting the brain, spinal cord, and associated tissues. As used herein, the term “anatomic area” refers to an area of CNS anatomy, i.e., anatomy including brain, spinal cord and associated tissues.

A CNS neoplasm is understood to be a tumor, either primary, recurrent or regionally metastatic, that affects a region of the body in the brain, spinal cord, and surrounding structures, including meninges, blood vessels, bone, and connective tissue. A primary tumor that has spread to regional sites is understood to be regionally metastatic; a tumor that has spread to distant sites beyond the locoregional area is said to be distantly metastatic. In certain embodiments, biocompatible and optionally biocompatible polymers may be used to allow for sustained release of an encapsulated antineoplastic taxane to treat a CNS neoplasm. The present invention also relates to methods of administering such pharmaceutical compositions, e.g., as part of a treatment regimen, for example, into tumors, into arteries or other vessels nourishing tumors, into the cerebrospinal fluid space, into an excised tumor bed, into the margins of an excised tumor, or in conjunction with a reconstructive tissue that is used to reconstruct a defect created by tumor extirpation or other local tumor control. The present invention also provides for kits whereby said pharmaceutical compositions may be delivered to the aforesaid sites.

In certain aspects, the subject compositions, upon contact with body fluids including blood, lymph, tissue fluid or the like, release the encapsulated antineoplastic taxane over a sustained or extended period (as compared to the release from an isotonic saline solution). Such a system may result in prolonged delivery (over, for example, 2 to 4,000 hours, or 4 to 1500 hours) of effective amounts (e.g., 0.00001 mg/kg/hour to 10 mg/kg/hour) of the drug. This dosage form may be administered as is necessary depending on the subject being treated, the severity of the affliction, the judgment of the prescribing physician, and the like.

For treatment of CNS neoplasms, the pharmaceutical compositions of the present invention are adapted for application to a preselected anatomic area, for example an area of local disease or an area with regional metastasis or micrometastasis, or an area with a significant likelihood of containing residual disease after excision, or an area with a significant likelihood of bearing regional micrometastases. In certain embodiments, the subject compositions of the present invention are understood to exert their effect in part by contact with a portion of the anatomic area being treated. Contact refers to a physical touching, either directly with the subject composition being applied without intervening barrier to the anatomic area being treated, or indirectly, where the subject composition is applied to or is formed on a surface of an interposed material, passing therethrough to come into direct contact with the anatomic area being treated. Contact, as used herein, includes those situations where the pharmaceutical compounds of the present invention are initially positioned to contact the anatomic area being treated, and those situations where the pharmaceutical compounds of the present invention are initially positioned in proximity to the anatomic area being treated without contacting it, and subsequently move, migrate, flow, spread or are transported to enter into contact with the anatomic area being treated. Contact may include partial contacts, wherein the pharmaceutical compounds only contact a portion of the anatomic area being treated, or the edge or periphery or margin of the anatomic area being treated. Contact of the pharmaceutical compounds with the anatomic area being treated occurs from a local rather than systemic administration of said compounds, as these terms are defined hereinafter. The composition may be formed as a flowable material, insertable into the anatomic area. A variety of devices and methods for inserting the composition into the preselected anatomic area will be familiar to practitioners of ordinary skill in the art, for example infusion, injection, topical application, spraying, painting, coating, formed gel placement, and others. The composition, alternatively, may be formed as a solid object implantable in the anatomic area, or as a film or mesh that may be used to cover a segment of the area. A variety of techniques for implanting solid objects in relevant anatomic areas will be likewise familiar to practitioners of ordinary skill in the art.

In certain embodiments, the present invention may include the use of a composition in the manufacture of a medicament to treat a CNS neoplasm, wherein said composition comprises a therapeutically effective amount of a composition comprising a biocompatible polymer and an antineoplastic agent appropriate for a CNS neoplasm, wherein said biocompatible polymer comprises a biocompatible polymer having phosphorous-based linkages.

2. Definitions

For convenience, before further description of the present invention, certain terms employed in the specification, examples, and appended claims are collected here. These definitions should be read in light of the remainder of the disclosure and understood as by a person of skill in the art. Also, the terms “including” (and variants thereof), “such as”, “e.g.” as used herein are non-limiting and are for illustrative purposes only.

The articles “a” and “an” are used herein to refer to one or to more than one (i.e. to at least one) of the grammatical object of the article. By way of example, “an element” means one element or more than one element.

The term “access device” is an art-recognized term and includes any medical device adapted for gaining or maintaining access to an anatomic area. Such devices are familiar to artisans in the medical and surgical fields. An access device may be a needle, a catheter, a cannula, a trocar, a tubing, a shunt, a drain, an endoscope, or any other device adapted for use in the cranial, spinal, and brain areas, or any other medical device suitable for entering or remaining positioned within the preselected anatomic area.

The terms “antineoplastic”, “antineoplastic agent” and “antineoplastic substance” are art-recognized and refer to therapeutic agents that prevent the development, maturation, or spread of CNS neoplasms or cells located in an anatomic area characterized by abnormal malignant growth. Examples of antineoplastic agents are set forth below. Certain antineoplastic agents (termed “Type I antineoplastic agents”) require the use of electromagnetic radiation in connection with their administration to produce a therapeutic effect or otherwise have an acceptable therapeutic index; treatment with this type of antineoplastic agent alone without electromagnetic radiation will not produce a therapeutic effect acceptable to one of skill in the art. In contrast, other antfineoplastfic agents (termed “Type II antineoplastic agents”) have a therapeutic effect and have an acceptable therapeutic index without the concomitant use of electromagnetic radiation. For example, paclitaxel is a Type II antineoplastic agent. As described in greater detail below, certain Type II antineoplastic agents, such as paclitaxel, may achieve different therapeutic effects when used in conjunction with electromagnetic radiation, with the timing and type of such radiation treatments possibly also having an affect on the treatment results. In certain embodiments, an antineoplastic agent used in a composition of the invention to treat CNS neoplasms is as effective or more effective than paclitaxel or docetaxel, or is at least within an order of magnitude as effective as paclitaxel or docetaxel, e.g., has an ED₅₀ less than ten times the ED₅₀ of paclitaxel or docetaxel.

The terms “biocompatible polymer” and “biocompatibility” when used in relation to polymers are art-recognized. For example, biocompatible polymers include polymers that are neither themselves toxic to the host (e.g., an animal or human), nor degrade (if the polymer degrades) at a rate that produces monomeric or oligomeric subunits or other byproducts at toxic concentrations in the host in certain embodiments of the present invention, biodegradation generally involves degradation of the polymer in an organism, e.g., into its monomeric subunits, which may be known to be effectively non-toxic. Intermediate oligomeric products resulting from such degradation may have different toxicological properties, however, or biodegradation may involve oxidation or other biochemical reactions that generate molecules other than monomeric subunits of the polymer. Consequently, in certain embodiments, toxicology of a biodegradable polymer intended for in vivo use, such as implantation or injection into a patient, may be determined after one or more toxicity analyses. It is not necessary that any subject composition have a purity of 100% to be deemed biocompatible; indeed, it is only necessary that the subject compositions be biocompatible as set forth above. Hence, a subject composition may comprise polymers comprising 99%, 98%, 97%, 96%, 95%, 90%, 85%, 80%, 75% or even less of biocompatible polymers, e.g., including polymers and other materials and excipients described herein, and still be biocompatible.

To determine whether a polymer or other material is biocompatible, it may be necessary to conduct a toxicity analysis. Such assays are well known in the art. One example of such an assay may be performed with live carcinoma cells, such as GT3TKB tumor cells, in the following manner: the sample is degraded in 1 M NaOH at 37° C. until complete degradation is observed. The solution is then neutralized with 1 M HCl. About 200 μL of various concentrations of the degraded sample products are placed in 96-well tissue culture plates and seeded with human gastric carcinoma cells (GT3TKB) at 104/well density. The degraded sample products are incubated with the GT3TKB cells for 48 hours. The results of the assay may be plotted as % relative growth vs. concentration of degraded sample in the tissue-culture well. In addition, polymers and formulations of the present invention may also be evaluated by well-known in vivo tests, such as subcutaneous implantations in rats to confirm that they do not cause significant levels of irritation or inflammation at the subcutaneous implantation sites.

The term “biodegradable” is art-recognized, and includes polymers, compositions and formulations, such as those described herein, that are intended to degrade during use. Biodegradable polymers typically differ from non-biodegradable polymers in that the former may be degraded during use. In certain embodiments, such use involves in vivo use, such as in vivo therapy, and in other certain embodiments, such use involves in vitro use. In general, degradation attributable to biodegradability involves the degradation of a biodegradable polymer into its component subunits, or digestion, e.g., by a biochemical process, of the polymer into smaller, non-polymeric subunits. In certain embodiments, two different types of biodegradation may generally be identified. For example, one type of biodegradation may involve cleavage of bonds (whether covalent or otherwise) in the polymer backbone. In such biodegradation, monomers and oligomers typically result, and even more typically, such biodegradation occurs by cleavage of a bond connecting one or more of subunits of a polymer. In contrast, another type of biodegradation may involve cleavage of a bond (whether covalent or otherwise) internal to side chain or that connects a side chain to the polymer backbone. For example, an antineoplastic taxane or other chemical moiety attached as a side chain to the polymer backbone may be released by biodegradation. In certain embodiments, one or the other or both generally types of biodegradation may occur during use of a polymer. As used herein, the term “biodegradation” encompasses both general types of biodegradation.

The degradation rate of a biodegradable polymer often depends in part on a variety of factors, including the chemical identity of the linkage responsible for any degradation, the molecular weight, crystallinity, biostability, and degree of cross-linking of such polymer, the physical characteristics of the implant, shape and size, and the mode and location of administration. For example, the greater the molecular weight, the higher the degree of crystallinity, and/or the greater the biostability, the biodegradation of any biodegradable polymer is usually slower. The term “biodegradable” is intended to cover materials and processes also termed “bioerodible”.

In certain embodiments, if the biodegradable polymer also has an antineoplastic taxane or other material associated with it, the biodegradation rate of such polymer may be characterized by a release rate of such materials. In such circumstances, the biodegradation rate may depend on not only the chemical identity and physical characteristics of the polymer, but also on the identity of any such material incorporated therein.

In certain embodiments, polymeric formulations of the present invention biodegrade within a period that is acceptable in the desired application. In certain embodiments, such as in vivo therapy, such degradation occurs in a period usually less than about five years, one year, six months, three months, one month, fifteen days, five days, three days, or even one day on exposure to a physiological solution with a pH between 6 and 8 having a temperature of between 25 and 37° C. In other embodiments, the polymer degrades in a period of between about one hour and several weeks, depending on the desired application.

The term “drug delivery device” is an art-recognized term and refers to any medical device suitable for the application of a drug or antineoplastic agent to a targeted organ or anatomic region. The term includes, without limitation, those formulations of the compositions of the present invention that release the antineoplastic agent into the surrounding tissues of an anatomic area. The term further includes those devices that transport or accomplish the instillation of the compositions of the present invention towards the targeted organ or anatomic area, even if the device itself is not formulated to include the composition. As an example, a needle or a catheter through which the composition is inserted into an anatomic area or into a blood vessel or other structure related to the anatomic area is understood to be a drug delivery device. As a further example, a stent or a shunt or a catheter that has the composition included in its substance or coated on its surface is understood to be a drug delivery device.

When used with respect to an antineoplastic agent or other material, the term “sustained release” is art-recognized. For example, a subject composition which releases a substance over time may exhibit sustained release characteristics, in contrast to a bolus type administration in which the entire amount of the substance is made biologically available at one time. For example, in particular embodiments, upon contact with body fluids including blood, tissue fluid, lymph or the like, the polymer matrices (formulated as provided herein and otherwise as known to one of skill in the art) may undergo gradual degradation (e.g., through hydrolysis) with concomitant release of any material incorporated therein, e.g., paclitaxel, for a sustained or extended period (as compared to the release from a bolus). This release may result in prolonged delivery of therapeutically effective amounts of any incorporated antineoplastic agent. Sustained release will vary in certain embodiments as described in greater detail below.

The term “delivery agent” is an art-recognized term, and includes molecules that facilitate the intracellular delivery of an antineoplastic agent or other material. Examples of delivery agents include: sterols (e.g., cholesterol) and lipids (e.g., a cationic lipid, virosome or liposome).

The term “microspheres” is art-recognized, and includes substantially spherical colloidal structures, e.g., formed from biocompatible polymers such as subject compositions, having a size ranging from about one or greater up to about 1000 microns. In general, “microcapsules”, also an art-recognized term, may be distinguished from microspheres, because microcapsules are generally covered by a substance of some type, such as a polymeric formulation. The term “microparticles” is art-recognized, and includes microspheres and microcapsules, as well as structures that may not be readily placed into either of the above two categories, all with dimensions on average of less than 1000 microns. If the structures are less than about one micron in diameter, then the corresponding art-recognized terms “nanosphere,” “nanocapsule,” and “nanoparticle” may be utilized. In certain embodiments, the nanospheres, nancapsules and nanoparticles have an average diameter of about 500, 200, 100, 50 or 10 nm.

A composition comprising microspheres may include particles of a range of particle sizes. In certain embodiments, the particle size distribution may be uniform, e.g., within less than about a 20% standard deviation of the median volume diameter, and in other embodiments, still more uniform or within about 10% of the median volume diameter.

The phrases “parenteral administration” and “administered parenterally” are art-recognized terms, and include modes of administration other than enteral and topical administration, such as injections, and include, without limitation, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrasternal injection and infusion.

The term “treating” is art-recognized and includes preventing a disease, disorder or condition from occurring in an animal which may be predisposed to the disease, disorder and/or condition but has not yet been diagnosed as having it; inhibiting the disease, disorder or condition, e.g., impeding its progress; and relieving the disease, disorder or condition, e.g., causing regression of the disease, disorder and/or condition. Treating the disease or condition includes ameliorating at least one symptom of the particular disease or condition, even if the underlying pathophysiology is not affected.

The term “fluid” is art-recognized to refer to a non-solid state of matter in which the atoms or molecules are free to move in relation to each other, as in a gas or liquid. If unconstrained upon application, a fluid material may flow to assume the shape of the space available to it, covering for example, the surfaces of an excisional site or the dead space left under a flap. A fluid material may be inserted or injected into a limited portion of a space and then may flow to enter a larger portion of the space or its entirety. Such a material may be termed “flowable.” This term is art-recognized and includes, for example, liquid compositions that are capable of being sprayed into a site; injected with a manually operated syringe fitted with, for example, a 23-gauge needle; or delivered through a catheter. Also included in the term “flowable” are those highly viscous, “gel-like” materials at room temperature that may be delivered to the desired site by pouring, squeezing from a tube, or being injected with any one of the commercially available injection devices that provide injection pressures sufficient to propel highly viscous materials through a delivery system such as a needle or a catheter. When the polymer used is itself flowable, a composition comprising it need not include a biocompatible solvent to allow its dispersion within a body cavity. Rather, the flowable polymer may be delivered into the body cavity using a delivery system that relies upon the native flowability of the material for its application to the desired tissue surfaces. For example, if flowable, a composition comprising polymers according to the present invention it can be injected to form, after injection, a temporary biomechanical barrier to coat or encapsulate internal organs or tissues, or it can be used to produce coatings for solid implantable devices. In certain instances, flowable subject compositions have the ability to assume, over time, the shape of the space containing it at body temperature.

Viscosity is understood herein as it is recognized in the art to be the internal friction of a fluid or the resistance to flow exhibited by a fluid material when subjected to deformation. The degree of viscosity of the polymer may be adjusted by the molecular weight of the polymer and other methods for altering the physical characteristics of a specific polymer will be evident to practitioners of ordinary skill with no more than routine experimentation. The molecular weight of the polymer used in the composition of the invention may vary widely, depending on whether a rigid solid state (higher molecular weights) desirable, or whether a fluid state (lower molecular weights) is desired.

The phrase “pharmaceutically acceptable” is art-recognized. In certain embodiments, the term includes compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” is art-recognized, and includes, for example, pharmaceutically acceptable materials, compositions or vehicles, such as a liquid or solid filler, diluent, excipient, solvent or encapsulating material, involved in carrying or transporting any subject composition from one organ, or portion of the body, to another organ, or portion of the body. Each carrier must be “acceptable” in the sense of being compatible with the other ingredients of a subject composition and not injurious to the patient. In certain embodiments, a pharmaceutically acceptable carrier is non-pyrogenic. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, sunflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “pharmaceutically acceptable salts” is art-recognized, and includes relatively non-toxic, inorganic and organic acid addition salts of compositions of the present invention, including without limitation, antineoplastic taxanes, excipients, other materials and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc and the like. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenethylamine; (trihydroxymethyl)aminoethane; and the like. See, for example, J. Pharm. Sci., 66:1-19 (1977).

A “patient,” “subject,” or “host” to be treated by the subject method may mean either a human or non-human animal, such as primates, mammals, and vertebrates.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if it is administered after manifestation of the unwanted condition, the treatment is therapeutic (i.e., it is intended to diminish, ameliorate, or stabilize the existing unwanted condition or side effects thereof).

The term “preventing”, when used in relation to a condition, such as a local recurrence, a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Electromagnetic radiation” as used in this specification includes, but is not limited to, radiation having the wavelength of 10⁻²⁰ to 10 meters. Particular embodiments of electromagnetic radiation of the present invention employ the electromagnetic radiation of: gamma-radiation (10⁻²⁰ to 10⁻¹³ m), x-ray radiation (10⁻¹¹ to 10⁻⁹ m), ultraviolet light (10 nm to 400 nm), visible light (400 nm to 700 nm), infrared radiation (700 nm to 1.0 mm), and microwave radiation (1 mm to 30 cm).

The phrases “systemic administration,” “administered systemically,” “peripheral administration” and “administered peripherally” are art-recognized, and include the administration of a subject composition or other material at a site remote from the disease being treated. Administration of an agent directly into, onto or in the vicinity of a lesion of the disease being treated, even if the agent is subsequently distributed systemically, may be termed “local” or “topical” or “regional” administration, other than directly into the central nervous system, e.g., by subcutaneous administration, such that it enters the patient's system and, thus, is subject to metabolism and other like processes.

The terms “therapeutic agent”, “drug”, “medicament” and “bioactive substance” are art-recognized and include molecules and other agents that are biologically, physiologically, or pharmacologically active substances that act locally or systemically in a patient or subject to treat a disease or condition, such as a CNS disease, or a CNS neoplasm. Such agents may be acidic, basic, or salts; they may be neutral molecules, polar molecules, or molecular complexes capable of hydrogen bonding; they may be prodrugs in the form of ethers, esters, amides and the like that are biologically activated when administered into a patient or subject. Antineoplastic agents are exemplary therapeutic agents.

The phrase “therapeutically effective amount” is an art-recognized term. In certain embodiments, the term refers to an amount of an antineoplastic agent or other therapeutic agent (such as an antineoplastic taxane) that, when incorporated into a polymer of the present invention, produces some desired effect at a reasonable benefit/risk ratio applicable to any medical treatment In certain embodiments, the term refers to that amount necessary or sufficient to eliminate, reduce or maintain (e.g., prevent the spread of) a tumor or other target of a particular therapeutic regimen. The effective amount may vary depending on such factors as the disease or condition being treated, the particular targeted constructs being administered, the size of the subject or the severity of the disease or condition. One of ordinary skill in the art may empirically determine the effective amount of a particular compound without necessitating undue experimentation.

In certain embodiments, a therapeutically effective amount of an antineoplastic agent, such as an antineoplastic taxane, for in vivo use will likely depend on a number of factors, including: the rate of release of the agent from the polymer matrix, which will depend in part on the chemical and physical characteristics of the polymer; the identity of the agent; the mode and method of administration; and any other materials incorporated in the polymer matrix in addition to the agent.

The term “ED₅₀” is art-recognized. In certain embodiments, ED₅₀ means the dose of a drug which produces 50% of its maximum response or effect, or alternatively, the dose which produces a pre-determined response in 50% of test subjects or preparations. The term “LD₅₀” is art-recognized. In certain embodiments, LD₅₀ means the dose of a drug which is lethal in 50% of test subjects. The term “therapeutic index” is an art-recognized term which refers to the therapeutic index of a drug, defined as LD₅₀/ED₅₀.

The terms “incorporated” and “encapsulated” are art-recognized when used in reference to an antineoplastic agent (or other material) and a polymeric composition, such as a composition of the present invention. In certain embodiments, these terms include incorporating, formulating or otherwise including such agent into a composition which allows for sustained release of such agent in the desired application. The terms may contemplate any manner by which an antineoplastic agent or other material is incorporated into a polymer matrix, including for example: attached to a monomer of such polymer (by covalent or other binding interaction) and having such monomer be part of the polymerization to give a polymeric formulation, distributed throughout the polymeric matrix, appended to the surface of the polymeric matrix (by covalent or other binding interactions), encapsulated inside the polymeric matrix, etc. The term “co-incorporation” or “co-encapsulation” refers to the incorporation of an antineoplastic agent or other material and at least one other antineoplastic agent or other material in a subject composition.

More specifically, the physical form in which any antineoplastic agent or other material is encapsulated in polymers may vary with the particular embodiment. For example, an antineoplastic agent or other material may be first encapsulated in a microsphere and then combined with the polymer in such a way that at least a portion of the microsphere structure is maintained. Alternatively, an antineoplastic agent or other material may be sufficiently immiscible in the polymer of the invention that it is dispersed as small droplets, rather than being dissolved, in the polymer. Any form of encapsulation or incorporation is contemplated by the present invention, in so much as the sustained release of any encapsulated antineoplastic agent or other material determines whether the form of encapsulation is sufficiently acceptable for any particular use.

The term “biocompatible plasticizer” is art-recognized, and includes materials which are soluble or dispersible in the compositions of the present invention, which increase the flexibility of the polymer matrix, and which, in the amounts employed, are biocompatible. Suitable plasticizers are well known in the art and include those disclosed in U.S. Pat. Nos. 2,784,127 and 4,444,933. Specific plasticizers include, by way of example, acetyl tri-n-butyl citrate (c. 20 weight percent or less), acetyl trihexyl citrate (c. 20 weight percent or less), butyl benzyl phthalate, dibutyl phthalate, dioctylphthalate, n-butyryl tri-n-hexyl citrate, diethylene glycol dibenzoate (c. 20 weight percent or less) and the like.

“Small molecule” is an art-recognized term. In certain embodiments, this term refers to a molecule which has a molecular weight of less than about 2000 amu, or less than about 1000 amu, and even less than about 500 amu.

The term “aliphatic” is an art-recognized term and includes linear, branched, and cyclic alkanes, alkenes, or alkynes. In certain embodiments, aliphatic groups in the present invention are linear or branched and have from 1 to about 20 carbon atoms.

The term “alkyl” is art-recognized, and includes saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 30 or fewer carbon atoms in its backbone (e.g., C₁-C₃₀ for straight chain, C₃-C₃₀ for branched chain), and alternatively, about 20 or fewer. Likewise, cycloalkyls have from about 3 to about 10 carbon atoms in their ring structure, and alternatively about 5, 6 or 7 carbons in the ring structure.

Moreover, the term “alkyl” (or “lower alkyl”) includes both “unsubstituted alkyls” and “substituted alkyls”, the latter of which refers to alkyl moieties having substituents replacing a hydrogen on one or more carbons of the hydrocarbon backbone. Such substituents may include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain may themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls may be further substituted with alkyls, alkenyls, alkoxys, alkylthios, aminoalkyls, carbonyl-substituted alkyls, —CF₃, —CN, and the like.

The term “aralkyl” is art-recognized, and includes alkyl groups substituted with an aryl group (e.g., an aromatic or heteroaromatic group).

The terms “alkenyl” and “alkynyl” are art-recognized, and include unsaturated aliphatic groups analogous in length and possible substitution to the alkyls described above, but that contain at least one double or triple bond respectively.

Unless the number of carbons is otherwise specified, “lower alkyl” refers to an alkyl group, as defined above, but having from one to ten carbons, alternatively from one to about six carbon atoms in its backbone structure. Likewise, “lower alkenyl” and “lower alkynyl” have similar chain lengths.

The term “heteroatom” is art-recognized, and includes an atom of any element other than carbon or hydrogen. Illustrative heteroatoms include boron, nitrogen, oxygen, phosphorus, sulfur and selenium, and alternatively oxygen, nitrogen or sulfur.

The term “aryl” is art-recognized, and includes 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring may be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocyclyls.

The terms ortho, meta and para are art-recognized and apply to 1,2-, 1,3- and 1,4-disubstituted benzenes, respectively. For example, the names 1,2-dimethylbenzene and ortho-dimethylbenzene are synonymous.

The terms “heterocyclyl” and “heterocyclic group” are art-recognized, and include 3- to about 10-membered ring structures, such as 3- to about 7-membered rings, whose ring structures include one to four heteroatoms. Heterocycles may also be polycycles. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrinidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring may be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” and “polycyclic group” are art-recognized, and include structures with two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms, e.g., three or more atoms are common to both rings, are termed “bridged” rings. Each of the rings of the polycycle may be substituted with such substituents as described above, as for example, halogen, alkyl, aralkyl alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl ketone, aldehyde, ester, a heterocyclyl an aromatic or heteroaromatic moiety, —CF₃, —CN, or the like.

The term “carbocycle” is art recognized and includes an aromatic or non-aromatic ring in which each atom of the ring is carbon. The flowing art-recognized terms have the following meanings: “nitro” means —NO₂; the term “halogen” designates —F, —Cl, —Br or —I; the term “sulfhydryl” means —SH; the term “hydroxyl” means —OH; and the term “sulfonyl” means —SO₂⁻.

The terms “amine” and “amino” are art-recognized and include both unsubstituted and substituted amines, e.g., a moiety that may be represented by the general formulas: 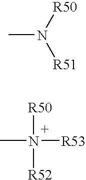
wherein R50, R51 and R52 each independently represent a hydrogen, an alkyl, an alkenyl, —(CH₂)ₘ—R61, or R50 and R51, taken together with the N atom to which they are attached complete a heterocycle having from 4 to 8 atoms in the ring structure; R61 represents an aryl, a cycloalkyl, a cycloalkenyl, a heterocycle or a polycycle; and m is zero or an integer in the range of 1 to 8. In certain embodiments, only one of R50 or R51 may be a carbonyl, e.g., R50, R51 and the nitrogen together do not form an imide. In other embodiments, R50 and R51 (and optionally R52) each independently represent a hydrogen, an alkyl, an alkenyl, or —(CH₂)ₘ—R61. Thus, the term “alkylamine” includes an amine group, as defined above, having a substituted or unsubstituted alkyl attached thereto, i.e., at least one of R50 and R51 is an alkyl group.

The term “acylamino” is art-recognized and includes a moiety that may be represented by the general formula: 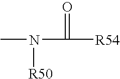
wherein R50 is as defined above, and R54 represents a hydrogen, an alkyl, an alkenyl or —(CH₂)ₘ—R61, where m and R61 are as defined above.

The term “amido” is art recognized as an amino-substituted carbonyl and includes a moiety that may be represented by the general formula: 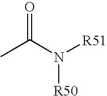
wherein R50 and R51 are as defined above. Certain embodiments of the amide in the present invention will not include imides which may be unstable.

The term “alkylthio” is art recognized and includes an alkyl group, as defined above, having a sulfur radical attached thereto. In certain embodiments, the “alkylthio” moiety is represented by one of —S-alkyl, —S-alkenyl, —S-alkynyl, and —S—(CH₂)ₘ—R61, wherein m and R61 are defined above. Representative alkylthio groups include methylthio, ethyl thio, and the like.

The term “carbonyl” is art recognized and includes such moieties as may be represented by the general formulas: 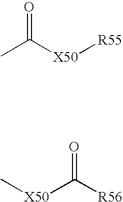
wherein X50 is a bond or represents an oxygen or a sulfur, and R55 represents a hydrogen, an alkyl, an alkenyl, —(CH₂)ₘ—R61 or a pharmaceutically acceptable salt, R56 represents a hydrogen, an alkyl, an alkenyl or —(CH₂)ₘ—R61, where m and R61 are defined above. Where X50 is an oxygen and R55 or R56 is not hydrogen, the formula represents an “ester”. Where X50 is an oxygen, and R55 is as defined above, the moiety is referred to herein as a carboxyl group, and particularly when R55 is a hydrogen, the formula represents a “carboxylic acid”. Where X50 is an oxygen, and R56 is hydrogen, the formula represents a “formate”. In general, where the oxygen atom of the above formula is replaced by sulfur, the formula represents a “thiocarbonyl” group. Where X50 is a sulfur and R55 or R56 is not hydrogen, the formula represents a “thioester.” Where X50 is a sulfur and R55 is hydrogen, the formula represents a “thiocarboxylic acid.” Where X50 is a sulfur and R56 is hydrogen, the formula represents a “thioformate.” On the other hand, where X50 is a bond, and R55 is not hydrogen, the above formula represents a “ketone” group. Where X50 is a bond, and R55 is hydrogen, the above formula represents an “aldehyde” group.

The terms “alkoxyl” or “alkoxy” are art recognized and include an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O—(CH₂)ₘR61, where m and R61 are described above.

The term “sulfonate” is art recognized and includes a moiety that may be represented by the general formula: 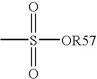
in which R57 is an electron pair, hydrogen, alkyl, cycloalkyl, or aryl.

The term “sulfate” is art recognized and includes a moiety that may be represented by the general formula: 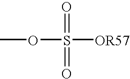
in which R57 is as defined above.

The term “sulfonamido” is art recognized and includes a moiety that may be represented by the general formula: 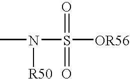
in which R50 and R56 are as defined above.

The term “sulfamoyl” is art-recognized and includes a moiety that may be represented by the general formula: 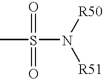
in which R50 and R51 are as defined above.

The term “sulfonyl” is art recognized and includes a moiety that may be represented by the general formula: 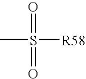
in which R58 is one of the following: hydrogen, alkyl, alkenyl, alkynyl, cycloalkyl, heterocyclyl, aryl or heteroaryl.

The term “sulfoxido” is art recognized and includes a moiety that may be represented by the general formula: 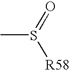
in which R58 is defined above.

The term “phosphoramidite” is art recognized and includes moieties represented by the general formulas: 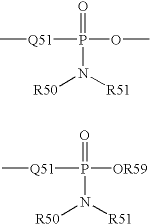
wherein Q51, R50, R51 and R59 are as defined above.

The term “phosphonamidite” is art recognized and includes moieties represented by the general formulas: 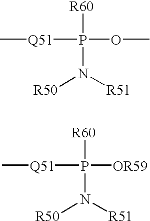
wherein Q51, R50, R51 and R59 are as defined above, and R60 represents a lower alkyl or an aryl.

Analogous substitutions may be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

The definition of each expression, e.g. alkyl, m, n, etc., when it occurs more than once in any structure, is intended to be independent of its definition elsewhere in the same structure unless otherwise indicated expressly or by the context.

The term “selenoalkyl” is art recognized and includes an alkyl group having a substituted seleno group attached thereto. Exemplary “selenoethers” which may be substituted on the alkyl are selected from one of —Se-alkyl, —Se-alkenyl, —Se-alkynyl, and —Se-(CH₂)ₘ—R61, m and R61 being defined above.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, and Ms are art recognized and represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations.

Certain monomeric subunits of the present invention may exist in particular geometric or stereoisomeric forms. In addition, polymers and other compositions of the present invention may also be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

For purposes of this invention, the chemical elements are identified in accordance with the Periodic Table of the Elements, CAS version, Handbook of Chemistry and Physics, 67th Ed., 1986-87, inside cover. The term “hydrocarbon” is art recognized and includes all permissible compounds having at least one hydrogen and one carbon atom. For example, permissible hydrocarbons include acyclic and cyclic, branched and unbranched, carbocyclic and heterocyclic, aromatic and nonaromatic organic compounds that may be substituted or unsubstituted.

The phrase “protecting group” is art recognized and includes temporary substituents that protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed. Greene et al., Protective Groups in Organic Synthesis 2^{nd} ed., Wiley, New York, (1991).

The phrase “hydroxyl-protecting group” is art recognized and includes those groups intended to protect a hydroxyl group against undesirable reactions during synthetic procedures and includes, for example, benzyl or other suitable esters or ethers groups known in the art.

The term “electron-withdrawing group” is recognized in the art, and denotes the tendency of a substituent to attract valence electrons from neighboring atoms, i.e., the substituent is electronegative with respect to neighboring atoms. A quantification of the level of electron-withdrawing capability is given by the Hammett sigma (a) constant. This well known constant is described in many references, for instance, March, Advanced Organic Chemistry 251-59, McGraw Hill Book Company, New York, (1977). The Hammett constant values are generally negative for electron donating groups (σ(P)=−0.66 for NH₂) and positive for electron withdrawing groups (σ(P)=0.78 for a nitro group), σ(P) indicating para substitution. Exemplary electron-withdrawing groups include nitro, acyl, formyl, sulfonyl, trifluoromethyl, cyano, chloride, and the like. Exemplary electron-donating groups include amino, methoxy, and the like.

Contemplated equivalents of the polymers, subunits and other compositions described above include such materials which otherwise correspond thereto, and which have the same general properties thereof (e.g., biocompatible, antineoplastic), wherein one or more simple variations of substituents are made which do not adversely affect the efficacy of such molecule to achieve its intended purpose. In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants which are in themselves known, but are not mentioned here.

3. Exemplary Subject Compositions

A variety of antineoplastic agents are contemplated by the present invention. Practitioners of the art will readily appreciate the circumstances under which various antineoplastic agents are appropriate for administration in the CNS and/or for treatment of a CNS neoplasm. For example, as described in the Exemplification section below, paclitaxel, an antineoplastic taxane, was used to treat CNS neoplasms.

Non-limiting examples of antineoplastic agents include, in general, microtubule-stabilising agents (such as paclitaxel, docetaxel or their derivatives or analogs); alkylating agents; anti-metabolites; epidophyllotoxin; an antineoplastic enzyme; a topoisomerase inhibitor; procarbazine; mitoxantrone; platinum coordination complexes; biological response modifiers and growth inhibitors; and haematopoietic growth factors. Exemplary classes of antineoplastic agents include, for example, the anthracycline family of drugs, the vinca drugs, the mitomycins, the bleomycins, the cytotoxic nucleosides, the taxanes, the epothilones, discodermolide, the pteridine family of drugs, diynenes and the podophyllotoxins. Members of those classes include, for example, doxorubicin, carminomycin, daunorubicin, aminopterin, methotrexate, methopterin, dichloro-methotrexate, mitomycin C, porfiromycin, trastuzumab (Herceptin.TM.), 5-fluorouracil, 6-mercaptopurine, gemcitabine, cytosine arabinoside, podophyllotoxin or podo-phyllotoxin derivatives such as etoposide, etoposide phosphate or teniposide, melphalan, vinblastine, vincristine, leurosidine, vindesine, leurosine, paclitaxel and the like. Other useful antineoplastic agents include estramustine, cisplatin, carboplatin, cyclophosphamide, bleomycin, gemcitibine, ifosamide, melphalan, hexamethyl melamine, thiotepa, cytarabin, idatrexate, trimetrexate, dacarbazine, L-asparaginase, camptothecin, CPT-11, topotecan, pyridobenzoindole derivatives, interferons and interleukins.

Still other representative antineoplastic agents include: alkylating agents such as nitrogen mustards, for instance mechlorethamine, cyclophosphamide, melphatan and chlorambucil, alkyl sulptronates such as busulphan, nitrosoureas such as carmustine, lomusine, semustine and streptozocin, triazenes such as dacarbazine, antimetabolites such as folic acid analogues, for instance methotrexate, pyrimidine analogues such as fluorouracil and cytarabine, purine analogues such as mercaptopurine and thioguanine, natural products such as vinca alkaloids, for instance vinblastine, vincristine and vendesine, epipodophyllotoxins such as etoposide and teniposide, antibiotics such as dactinomycin, daunorubicin, doxorubicin, bleomycin, plicamycin and mitomycin, enzymes such as L-asparaginase, substituted ureas such as hydroxyurea, methylhydrazine derivatives such as procarbazine, adrenocorticoid suppressants such as mitotane and aminoglutethimide, hormones and antagonists such as adrenocorticosteroids such as prednisone, progestins such as hydroxyprogesterone caproate, methoxyprogesterone acetate and megestrol acetate, oestrogens such as diethylstilboestrol and ethinyloestradiol, antioestrogens such as tamoxifen, and androgens such as testosterone propionate and fluoxymesterone.

In certain embodiments, the antineoplastic agent is a member of the class of agents hereinafter defined as the antineoplastic taxanes, of which paclitaxel and docetaxel are two members. Paclitaxel and docetaxel share a common framework, and differ primarily in the substituents at two sites on this framework, shown as R1 and R2 in Formula I below:

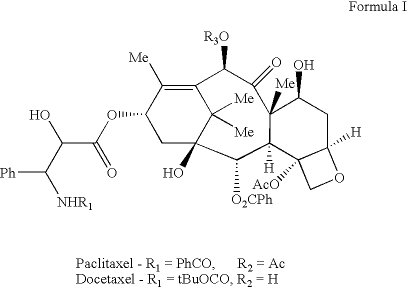

Thus, in one embodiment, a therapeutic composition of the invention comprises a compound of the above formula, wherein R1 is an acyl group or R1-N taken together comprise a carbamyl group (O—C(═O)—N), and R2 is H or an acyl group. In particular embodiments, R1 comprises between 2 and 12 carbon atoms, or between 4 and 9 carbon atoms. In some embodiments, R2 is H or an acyl group having between 2 and 8 carbons, or between 2 and 4 carbons. In certain embodiments, the antineoplastic taxane is docetaxel or paclitaxel.

In another embodiment, a therapeutic composition of the present invention includes an antineoplastic agent having a structure of Formula II: 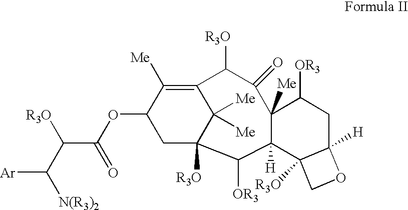
wherein, independently for each occurrence:
Ar represents a substituted or unsubstituted aryl or heteroaryl group; and
R3, each independently, represents H, alkyl, acyl, alkoxycarbonyl, aryloxycarbonyl, aminocarbonyl or sulfonyl.

In certain embodiments, at least one R3 is bound to nitrogen is H or alkyl. In certain embodiments, at least one R3 bound to nitrogen is acyl, alkoxycarbonyl, aryloxycarbonyl, aminocarbonyl, or sulfonyl. In certain embodiments, when R3 is bound to oxygen, R3 is selected from H, alkyl, acyl, aminocarbonyl, alkoxycarbonyl, or aryloxycarbonyl. An R3 can be selected to be sterically similar to a corresponding substituent on paclitaxel or docetaxel, i.e., contains a number of carbon atoms within four of the number of carbon atoms in a similarly situated substituent of paclitaxel or docetaxel. For example, the benzoate ester of paclitaxel may be exchanged for a tosyl (p-toluenesulfonyl) ester, a cyclohexyl carbamate, or a tetrachlorobenzocyclopentanol carbonate, or a hydroxyl of docetaxel may be exchanged for an ethyl ether, a methylsulfonate ester, or a 2-hydroxyethyl carbamate.

In yet another embodiment, a therapeutic composition of the present invention includes an antineoplastic agent having a structure of Formula III: 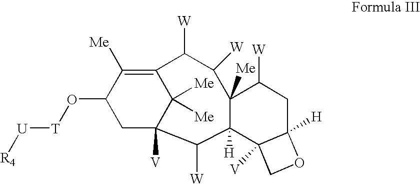
wherein, independently for each occurrence:
V, each independently, represents H, hydroxy, lower alkoxy, or a small ester (e.g., less than 4 carbons);
W, each independently, represents H, hydroxy, carbonyl, amino, alkoxy, sulfhydryl, alkylthio, ester, acylamino, carbamate, sulfonate, carbonate, or sulfoxide;
T represents —C(═O)—, —C(═S)—, —SO₂—, or —SO—;
U is absent or represents NH, S, or O; and

R4 represents a substituted aralkyl.

In certain embodiments, at least one occurrence of W or R4 includes a moiety, such as an oligopeptide or an oligosaccharide, that improves the bioavailability and/or solubility of the taxane. In certain embodiments, the therapeutic compound is formulated as a prodrug, e.g., at least one occurrence of W or R4 includes a moiety capable of being hydrolyzed and cleaved from the molecule under physiological conditions. The hydrolyzable moiety may improve the bioavailability and/or solubility of the taxane. The prodrug form of the therapeutic compound may itself be inactive, provided that after cleavage of the hydrolyzable moiety, the resulting compound is antineoplastic. In certain embodiments, at least one occurrence of W or R4 includes a bond to a polymer. The bond to the polymer may be hydrolyzable under physiologic conditions.

In certain embodiments, a therapeutic composition of the present invention includes an “antineoplastic taxane”, i.e., a compound which has a framework of Formula IV: 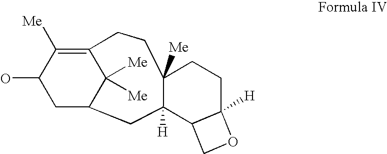
wherein, such framework bears sufficient substituents disposed at unspecified positions, as valence allows, such that the resulting compound has antineoplastic activity. In certain embodiments, such a compound is formed by chemically modifying paclitaxel or 10-deacetylbaccatin III, a naturally occurring compound which has the structure:

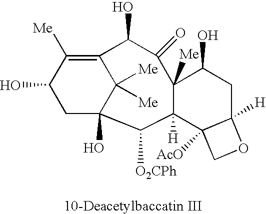

A variety of such antineoplastic derivatives are known in the art, and may be employed in the subject compositions and methods without departing from the spirit or scope of the present invention.

Still other antineoplastic agents will be known by those of skill in the art and may be encapsulated in the subject compositions without undue experimentation.

Polymers

A variety of polymers may be used in the subject invention. Both non-biodegradable and biodegradable polymers may be used in the subject invention. In some embodiments, biodegradable polymers may be used. As discussed below, the choice of polymer will depend in part on a variety of physical and chemical characteristics of such polymer and the use to which such polymer may be put.

Representative natural polymers include proteins, such as zein, modified zein, casein, gelatin, gluten, serum albumin, or collagen, and polysaccharides, such as cellulose, dextrans, hyaluronic acid, and polymers of alginic acid.

Representative synthetic polymers include polyphosphazines, poly(vinyl alcohols), polyamides, polycarbonates, polyalkylenes, polyacrylamides, polyanhydrides, poly(phosphoesters), polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyphosphates and polyurethanes.

Synthetically modified natural polymers include alkyl celluloses, hydroxyalkyl celluloses, cellulose ethers, cellulose esters, and nitrocelluloses. Other like polymers of interest include, but are not limited to, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate and cellulose sulfate sodium salt.

Representative biodegradable polymers include polylactide, polyglycolide, polycaprolactone, polycarbonate, poly(phosphoesters), polyanhydride, polyorthoesters, and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins.

All of the subject polymers may be provided as copolymers or terpolymers. These polymers may be obtained from chemical suppliers or else synthesized from monomers obtained from these suppliers using standard techniques.

In addition to the listing of polymers above, polymers having phosphorus linkages may be used in the subject invention. Exemplary phosphorus linkages in such polymers include, without limitation, phosphonamidite, phosphoramidite, phosphorodiamidate, phosphomonoester, phosphodiester, phosphotriester, phosphonate, phosphonate ester, phosphorothioate, thiophosphate ester, phosphinate or phosphite. Certain of such polymers may be biodegradable, biocompatible or both.

The structure of certain of the foregoing polymers having phosphorus linkages may be identified as follows. The term “polymer having phosphorous-based linkages” is used herein to refer to polymers in which the following substructure is present at least a multiplicity of times in the backbone of such polymer: 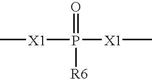
wherein, independently for each occurrence of such substructure:
   X1, each independently, represents —O— or —N(R5)-;
   R5 represents —H, aryl, alkenyl or alkyl; and
   R6 is any non-interfering substituent,
wherein such substructure is responsible in part for biodegradability properties, if any, observed for such polymer in vitro or in vivo. In certain embodiments, R6 may represent an alkyl, aralkyl, alkoxy, alkylthio, or alkylamino group.

In certain embodiments, such a biodegradable polymer is non-naturally occurring, i.e., a man-made product with no natural source. In other embodiments, R6 is other than —OH or halogen, e.g., is alkyl, aralkyl, aryl, alkoxy, aralkyoxy or aryloxy. In still other embodiments, the two X1 moieties in such substructure are the same. For general guidance, when reference is made to the “polymer backbone chain” or the like of a polymer, with reference to the above structure, such polymer backbone chain comprises the motif [-X1-P-X1-]. In other polymers, the polymer backbone chain may vary as recognized by one of skill in the art.

By way of example, but not limitation, a number of representative polymers having phosphorus linkages are described in greater detail below. In certain embodiments, a polymer includes one or more monomeric units of Formula V: 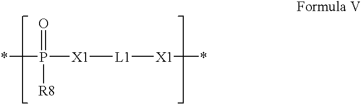
wherein, independently for each occurrence of such unit:
X1, each independently, represents —O— or —N(R7)—;
R7 represents —H, aryl, alkenyl or alkyl;
L1 is described below;
R8 represents, for example, —H, alkyl, —O-alkyl, —O-cycloalkyl, aryl, —O-aryl, heterocycle, —O-heterocycle, N(R9)R10 and other examples presented below;
R9 and R10, each independently, represent a hydrogen, an alky, an alkenyl, —(CH2)m—R11, or R9 and R10, taken together with the N atom to which they are attached complete a heterocycle having from 4 to about 8 atoms in the ring structure;
m represents an integer in the range of 0-10, or 0-6; and
R11 represents —H, alkyl, aryl, cycloalkyl, cycloalkenyl, heterocycle or polycycle.

L1 may be any chemical moiety as long as it does not materially interfere with the polymerization biocompatibility or biodegradation (or any combination of those three properties) of the polymer, wherein a “material interference” or “non-interfering substituent” is understood to mean: (i) for synthesis of the polymer by polymerization, an inability to prepare the subject polymer by methods known in the art or taught herein, (ii) for biocompatibility, a reduction in the biocompatibility of the subject polymer so as to make such polymer impracticable for in vivo use; and (iii) for biodegradation, a reduction in the biodegradation of the subject polymer so as to make such polymer impracticable for biodegradation.

In certain embodiments, L1 is an organic moiety, such as a divalent branched or straight chain or cyclic aliphatic group or divalent aryl group, with in certain embodiments, from 1 to about 20 carbon atoms. In certain embodiments, L1 represents a moiety between about 2 and 20 atoms selected from carbon, oxygen, sulfur, and nitrogen, wherein at least 60% of the atoms are carbon. In certain embodiments, L1 may be an alkylene group, such as methylene, ethylene, 1,2-dimethylethylene, n-propylene, isopropylene, 2,2-dimethylpropylene, n-pentylene, n-hexylene, n-heptylene; an alkenylene group such as ethenylene, propenylene, 2-(3-propenyl)-dodecylene; and an alkynylene group such as ethynylene, proynylene, 1-(4-butynyl)-3-methyldecylene; and the like. Such unsaturated aliphatic groups may be used to cross-link certain embodiments of the present invention.

Further, L1 may be a cycloaliphatic group, such as cyclopentylene, 2-methylcyclopentylene, cyclohexylene, cyclohexylenedimethylene, cyclohexenylene and the like. L1 may also be a divalent aryl group, such as phenylene, benzylene, naphthalene, phenanthrenylene and the like. Further, L1 may be a divalent heterocyclic group, such as pyrrolylene, furanylene, thiophenylene, alkylyene-pyrrolylene-alkylene, pyridinylene, pyrimidinylene and the like.

Other examples of L1 may include any of the polymers listed above, including the biodegradable polymers listed above, and in particular polylactide, polyglycolide, polycaprolactone, polycarbonate, polyethylene terephthalate, polyanhydride and polyorthoester, and polymers of ethylene glycol, propylene glycol and the like. Embodiments containing such polymers for L1 may impart a variety of desired physical and chemical properties.

The foregoing, as with other moieties described herein, may be substituted with a non-interfering substituent, for example, a hydroxy-, halogen-, or nitrogen-substituted moiety.

R8 represents hydrogen, alkyl, cycloakyl, —O-alkyl, —O-cycloalkyl, aryl, —O-aryl, heterocycle, —O-heterocycle, or —N(R9)R10. Examples of possible alkyl R8 groups include methyl, ethyl, n-propyl, i-propyl, n-butyl, tert-butyl, —C₈H₁₇ and the like groups; and alkyl substituted with a non-interfering substituent, such as hydroxy, halogen, alkoxy or nitro; corresponding alkoxy groups.

When R8 is aryl or the corresponding aryloxy group, it typically contains from about 5 to about 14 carbon atoms, or about 5 to about 12 carbon atoms, and optionally, may contain one or more rings that are fused to each other. Examples of particularly suitable aromatic groups include phenyl, phenoxy, naphthyl, anthracenyl, phenanthrenyl and the like.

When R8 is heterocyclic or heterocycloxy, it typically contains from about 5 to about 14 ring atoms, alternatively from about 5 to about 12 ring atoms, and one or more heteroatoms. Examples of suitable heterocyclic groups include furan, thiophene, pyrrole, isopyrrole, 3-isopyrrole, pyrazole, 2-isoimidazole, 1,2,3-triazole, 1,2,4-triazole, oxazole, thiazole, isothiazole, 1,2,3-oxadiazole, 1,2,4-oxadiazole, 1,2,5-oxadiazole, 1,3,4-oxadiazole, 1,2,3,4-oxatriazole, 1,2,3,5-oxatriazole, 1,2,3-dioxazole, 1,2,4-dioxazole, 1,3,2-dioxazole, 1,3,4-dioxazole, 1,2,5-oxatriazole, 1,2-pyran, 1,4-pyran, 1,2-pyrone, 1,4-pyrone, 1,2-dioxin, 1,3-dioxin, pyridine, N-alkyl pyridinium, pyridazine, pyrimidine, pyrazine, 1,3,5-triazine, 1,2,4-triazine, 1,2,3-triazine, 1,2-oxazine, 1,3-oxazine, 1,4-oxazine, o-isoxazine, p-isoxazine, 1,2,5-oxathiazine, 1,2,6-oxathiazine, 1,4,2-oxadiazine, 1,3,5-oxadiazine, azepine, oxepin, thiepin, indene, isoindene, benzofuran, isobenzofuran, thionaphthene, isothionaphthene, indole, indolenine, 2-isobenzazole, isoindazole, indoxazine, benzoxazole, anthranil, 1,2-benzopyran, 1,2-benzopyrone, 1,4-benzopyrone, 2,1-benzopyrone, 2,3-benzopyrone, quinoline, isoquinoline, 12, -benzodiazine, 1,3-benzodiazine, naphthyridine, pyrido-[3,4-b]-pyridine, pyrido-[3,2-b]-pyridine, pyrido-[4,3-b]-pyridine, 1,3,2-benzoxazine, 1,4,2-benzoxazine, 2,3,1-benzoxazine, 3,1,4-benzoxazine, 1,2-benzisoxazine, 1,4-benzisoxazine, carbazole, xanthrene, acridine, purine, and the like. In certain embodiments, when R8 is heterocyclic or heterocycloxy, it is selected from the group consisting of furan, pyridine, N-alkylpyridine, 1,2,3- and 1,2,4-triazoles, indene, anthracene and purine rings.

In certain embodiments, R8 is an alkyl group, an alkoxy group, a phenyl group, a phenoxy group, a heterocycloxy group, or an ethoxy group.

In still other embodiments, R8, such as an alkyl, may be conjugated to a bioactive substance to form a pendant drug delivery system.

In certain embodiments, the number of monomeric units in Formula V and other subject formulas that make up the subject polymers ranges over a wide range, e.g., from about 2, 3, 4, 5 to 25,000 or more, but generally from about 50, 100 to 5000, or 10,000. Alternatively, in other embodiments, the number of monomeric units may be about 10, 25, 50, 75, 100, 150, 200, 300 or 400.

In Formula V and other formulas herein, “*” represents other monomeric units of the subject polymer, which may be the same or different from the unit depicted in the formula in question, or a chain terminating group, by which the polymer terminates. Examples of such chain terminating groups include monofunctional alcohols and amines.

In another aspect, the polymeric compositions of the present invention include one or more recurring monomeric units represented in general Formula VI: 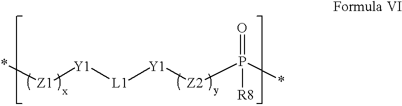
wherein Z1 and Z2, respectively, for each independent occurrence is: 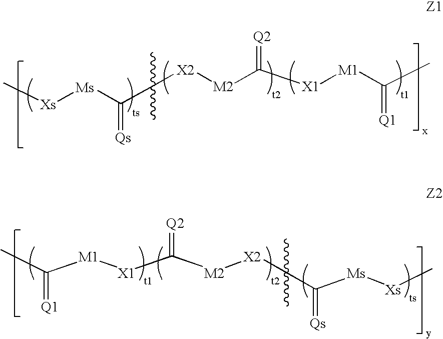
wherein, independently for each occurrence set forth above:
   Q1, Q2 . . . . Qs, each independently, represent O or N(R1);
   X1, X2. Xs, each independently, represent —O— or —N(R1);
   the sum of t1, t2 . . . ts is an integer and at least one or more;
   Y1 represents —O—, —S— or —N(R7)—;
   x and y are each independently integers from 1 to about 1000 or more;
   L1 and M1, M2 . . . Ms each independently, represent the moieties discussed below; and
   the other moieties are as defined above.

M1, M2 . . . Ms (collectively, M) in Formula VI are each independently any chemical moiety that does not materially interfere with the polymerization, biocompatibility or biodegradation (or any combination of those three properties) of the subject polymer. For certain embodiments, M in the formula are each independently: (i) a branched or straight chain aliphatic or aryl group having from 1 to about 50 carbon atoms, or (ii) a branched or straight chain, oxa-, thia-, or aza-aliphatic group having from 1 to about 50 carbon atoms, both optionally substituted. In certain embodiments, the number of such carbon atoms does not exceed 20. In other embodiments, M may be any divalent aliphatic moiety having from 1 to about 20 carbon atoms, including therein from 1 to about 7 carbon atoms.

M may include an aromatic or heteroaromatic moiety, optionally with non-interfering substituents. In certain embodiments, none of the atoms (usually but not always C) that form the cyclic ring that gives rise to the aromatic moiety are part of the polymer backbone chain.

Specifically, when M is a branched or straight chain aliphatic group having from 1 to about 20 carbon atoms, it may be, for example, an alkylene group such as methylene, ethylene, 1-methylethylene, 1,2-dimethylethylene, n-propylene, trimethylene, isopropylene, 2,2-dimethylpropylene, n-pentylene, n-hexylene, n-heptylene, n-octylene, n-nonylene, n-decylene, n-undecylene, n-dodecylene, and the like; an alkenylene group such as n-propenylene, 2-vinylpropylene, n-butenylene, 3-thexylbutylene, n-pentenylene, 4-(3-propenyl)hexylene, n-octenylene, 1-(4-butenyl)-3-methyldecylene, 2-(3-propenyl)dodecylene, hexadecenylene and the like; an alkynylene group, such as ethynylene, propynylene, 3-(2-ethynyl)pentylene, n-hexynylene, 2-(2-propynyl)decylene, and the like; or any alkylene, alkenylene or alkynylene group, including those listed above, substituted with a materially non-interfering substituent, for example, a hydroxy, halogen or nitrogen group, such as 2-chloro-n-decylene, 1-hydroxy-3-ethenylbutylene, 2-propyl-6-nitro-10-dodecynylene, and the like. Other M of the present invention include —CH₂)₃—, —(CH₂)₅— and —(CH₂)₂OCH₂—.

When M is a branched or straight chain oxaaliphatic group having from 1 to about 20 carbon atoms, it may be, for example, a divalent alkoxylene group, such as ethoxylene, 2-methylethoxylene, propoxylene, butoxylene, pentoxylene, dodecyloxylene, hexadecyloxylene, and the like. When M is a branched or straight chain oxaaliphatic group, it may have the formula —(CH₂)ₙ—O—(CH₂)_{b}— wherein each of a and b, independently, is about 1 to about 7.

When M is a branched or straight chain oxaaliphatic group having from 1 to about 20 carbon atoms, it may also be, for example, a dioxaalkylene group such as dioxymethylene, dioxyethylene, 1,3-dioxypropylene, 2-methoxy-1,3-dioxypropylene, 1,3-dioxy-2-methylpropylene, dioxy-n-pentylene, dioxy-n-octadecylene, methoxylene-methoxylene, ethoxylene-methoxylene, ethoxylene-ethoxylene, ethoxylene-1-propoxylene, butoxylene-n-propoxylene, pentadecyloxylene-methoxylene, and the like. When M is a branched or straight chain, dioxyaliphatic group, it may have the formula —(CH₂)ₐ—O—(CH₂)_{b}—O—(CH₂)_{c}-, wherein each of a, b, and c is independently from 1 to about 7.

When M is a branched or straight chain thiaaliphatic group, the group may be any of the preceding oxaaliphatic groups wherein the oxygen atoms are replaced by sulfur atoms.

When M is a branched or straight chain, aza-aliphatic group having from 1 to about 20 carbon atoms, it may be a divalent group such as —CH₂NH—, —(CH₂)₂N—, —CH₂(C₂H₅)N—, -n-C₄H₉NH—, -t-C₄H₉NH—, —CH₂(C₃H₇)N—, —C₂H₅(C₂H₅)N—, —CH₂(C₈H₁₇)N—, —CH₂NHCH₂—, —(CH₂)₂NCH₂—, —CH₂(C₂H₅)NCH₂CH₂—, -n-C₄H₉NHCH₂—, -t-C₄H₉NHCH₂CH₂—, —CH₂(C₃H₇)N(CH₂)₄—, —C₂H₅(C₂H₅)NCH₂—, —CH₂(C₈H₁₇)NCH₂CH₂—, and the like. When M is a branched or straight chain, amino-aliphatic group, it may have the formula —(CH₂)ₙNR1- or —(CH₂)ₐN(R1)(CH₂)_{b}— where R1 is —H, aryl, alkenyl or alkyl and each of a and b is independently from about 1 to about 7.

x and y of Formula VI each independently represent integers in the range of about 1 to about 1000, e.g., about 1, about 10, about 20, about 50, about 100, about 250, about 500, about 750, about 1000, etc.

For Formula VI, the average molar ratio of (x or y):L1, assuming ts is equal to one, may vary greatly, typically between about 75:1 and about 2:1. In certain embodiments, the average molar ratio of (x or y):L1, when ts is equal to one, is about 10:1 to about 4:1, or in some embodiments, about 5:1. The molar ratio of x:y may also vary; typically, such ratio is about 1. Other possible embodiments may have ratios of 0.1, 0.25, 0.5, 0.75, 1.5, 2, 3, 4, 10 and the like.

A number of different polymer structures are contemplated by Formula VI. For example, in certain polymers exemplified by Formula VI, when the sum of t2 . . . ts equals one for each of Z1 and Z2 and Q, M and X for each subunit ts are the same, then Formula VI becomes the following Formula Via:

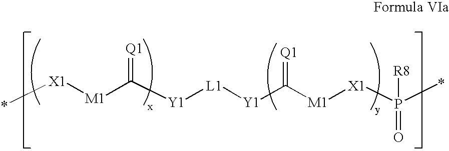

In certain embodiments of Formula VIa (and other subject formulas), x and y may be even integers.

The above Formula VI (and all of the subject formulae and polymers) encompass a variety of different polymer structures, including block copolymers, random copolymers, random terpolymers and segmented block copolymers and terpolymers. Additional structures for Z of subject monomeric units are set forth below, which exemplify in part the variety of structures contemplated by the present invention:

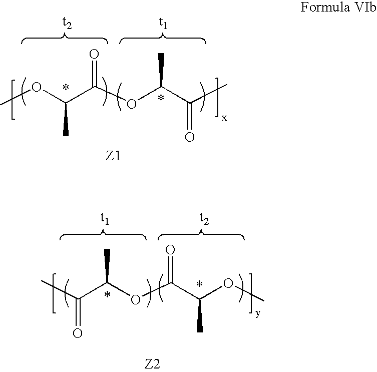

In Formula VIb (and other formulas described below), there may be more ts subunits depicted of the same molecular identity of those depicted in the formulas. For example, in Formula VIb, subunits t₁ and t₂ may be repeated in a sequence, e.g., alternating, in blocks (which may themselves repeat), or in any other pattern or random arrangement. Each subunit may repeat any number of times, and one subunit (e.g., t₁) may occur with substantially the same frequency, more often, or less often than another subunit (e.g., t₂), such that both subunits may be present in approximately the same amount, or in differing amounts, which may differ slightly or be highly disparate, e.g., one subunit is present nearly to the exclusion of the other. In certain embodiments, the chiral centers of each subunit may be the same or different and may be arranged in an orderly fashion or in a random sequence in each of Z1 and Z2.

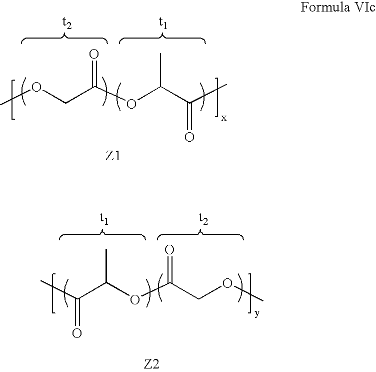

In certain embodiments of Formula VIc, the sum of the number of ts subunits in each of Z1 and Z2 is an even integer. As in other examples of Z1 and Z2, such as described above for Formula VIb, the ts subunits may be distributed randomly or in an ordered arrangement in each of Z1 or Z2.

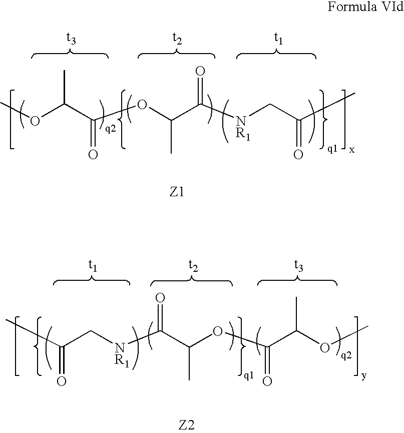

In Formula VId, the subunit q1 is comprised of two ts subunits, which may be repeated and arranged as described above for Formula VIb. In certain embodiments, q2 is an even integer, and in other embodiments, the subunits q1 and q2 may be distributed randomly or in an ordered pattern in each of Z1 and Z2. For example, subunits q1 and q2 may be repeated in a sequence, e.g., alternating, in blocks (which may themselves repeat), or in any other pattern or random arrangement. Each subunit may repeat any number of times, and one subunit (e.g., q1) may occur with substantially the same frequency, more often, or less often than another subunit (e.g., q2), such that both subunits may be present in approximately the same amount, or in differing amounts, which may differ slightly or be highly disparate, e.g., one subunit is present nearly to the exclusion of the other.

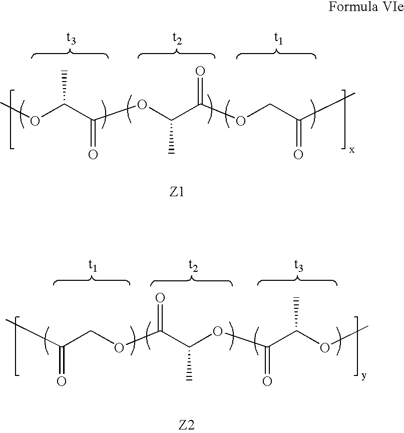

In certain embodiments of Formula VIe, the sum of the ts subunits for each of Z1 and Z2 is an even integer. In other embodiments, the each of the subunits t₁, t₂, and t₃ may be distributed randomly or in an ordered arrangement in each of Z1 and Z2. For example, in Formula VIe, subunits t₁, t₂, and t₃ may be repeated in a sequence, e.g., alternating, in blocks (which may themselves repeat), or in any other pattern or random arrangement. Each subunit may repeat any number of times, and one subunit (e.g., t₁) may occur with substantially the same frequency, more often, or less often than another subunit (e.g., t₃), such that the three subunits may be present in approximately the same amount, or in differing amounts, which may differ slightly or be highly disparate, e.g., two subunits are present nearly to the exclusion of the third.

In certain embodiments of Formula VI, in which Q, M and X for each subunit are the same, Q1 represents O, M represents a lower alkylene group, and X1 represents O or S. In some embodiment, X1 is O. For example, M may represent —CH(CH₃)— to result in a polymer of Formula VI having a structure represented in Formula VIf:

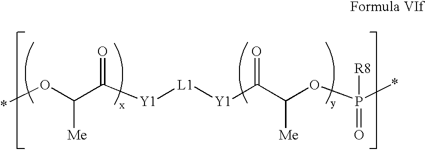

In certain embodiments of Formula VIf, as further described in the Exemplification below, L1 represents a lower alkylene chain, such as ethylene, propylene, etc. In certain embodiments, all Y1's represent O. In certain embodiments, R8 represents —O-lower alkyl, such as —OEt.

In certain embodiments of polymers depicted by Formula VI, the chirality of each subunit is identical, whereas in other embodiments, the chirality is different. By way of example but not limitation, in Formula VIb above, if the chiral centers of all of the subunits are D-enantiomers or L-enantiomers, then the monomeric unit is effectively equivalent to D-lactic acid or L-lactic acid, respectively, thereby giving rise to a region similar to poly(D-lactic acid) or poly(L-lactic acid), respectively. Conversely, if the two subunits in Formula VIb are comprised of alternating D- and L-enantiomers (e.g., one unit of D-enantiomer, one unit of L-enantiomer, etc.), then the resulting polymeric region is analogous to poly(meso-lactic acid) (i.e., a polymer formed by polymerization of meso-lactide).

Finally, in certain embodiments of the monomeric units set forth in Formula VI, in which the entire polymer may or may not be composed of such units, the following moieties for Y1, L1, R8 Qs, Xs and Ms may be used (with a variety of different x and y being possible):

| Abbreviation | All Y1's | L1 | R8 |
|---|---|---|---|
| _{L}-PL(EG)EOP | O | —CH₂CH₂— | —OCH₂CH₃ |
| _{L}PL(EG)HOP | O | —CH₂CH₂— | —O(CH₂)₅CH₃ |
| _{D,L}-PL(EG)EOP* | O | —CH₂CH₂— | —OCH₂CH₃ |
| _{D,L}-PL(PG)EOP* | O | —CH₂(CH₃)CH₂— | —OCH₂CH₃ |
| _{D}-PL(PG)EOP | O | —CH₂(CH₃)CH₂— | —OCH₂CH₃ |
| _{L}-PL(PG)EOP | O | —CH₂(CH₃)CH₂— | —OCH₂CH₃ |
| _{D,L}-PL(HD)EOP* | O | 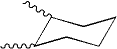 | —OCH₂CH₃ |
| _{D,L}-PL(PG)HOP* | O | —CH₂(CH₃)CH₂— | —O(CH₂)₅CH₃ |
| _{D,L}-PL(PG)EP* | O | —CH₂(CH₃)CH₂— | —CH₂CH₃ |

| | All | All | | |
|---|---|---|---|---|
| Abbreviation | Qs | Xs | M1 | M2 |
| _{L}-PL(EG)EOP | O | O | —CH(CH₃)-(_{L}) | N/A |
| _{L}-PL(EG)HOP | O | O | —CH(CH₃)-(_{L}) | N/A |
| _{D,L}-PL(EG)EOP* | O | O | —CH(CH₃)-(_{L or D}) | —CH(CH₃)-(_{D or L}) |
| _{D,L}-PL(PG)EOP* | O | O | —CH(CH₃)-(_{L or D}) | —CH(CH₃)-(_{D or L}) |
| _{D}-PL(PG)EOP | O | O | —CH(CH₃)-(_{D}) | N/A |
| _{L}-PL(PG)EOP | O | O | —CH(CH₃)-(_{L}) | N/A |
| _{D,L}-PL(HD)EOP* | O | O | —CH(CH₃)-(_{L or D}) | —CH(CH₃)-(_{L or D}) |
| _{D,L}-PL(PG)HOP* | O | O | —CH(CH₃)-(_{L or D}) | —CH(CH₃)-(_{L or D}) |
| _{D,L}-PL(PG)EP* | O | O | —CH(CH₃)-(_{L or D}) | —CH(CH₃)-(_{L or D}) |

*For _{D,L}-PL(EG)EOP, _{D,L}-PL(PG)EOP, _{D,L}-PL(HD)EOP, _{D,L}-PL(PG)HOP, and _{D,L}-PL(PG)EP, if the chiral carbon of M1 has configuration L, then M2 will have configuration D, and vice-versa. The order of the chiral centers in each subunit M1 and M2 for each Z1 and Z2 will be in random order.

In addition to the particular chiral version of the subject polymers described in the above table, polymers in which the chirality of MS varies in each subunit M in the subject polymers are also possible. For instance, referring to D,L-PL(EG)EOP by example, a random order of D and L, in varying amounts, are possible for this polymer. In contrast, the table sets forth one such example in which a D and L chiral M are always adjacent, in equal amounts, but that need not always be the case.

In another embodiment of the present invention, the polymeric compositions of the present invention include one or more recurring monomeric units represented in general Formula VII: 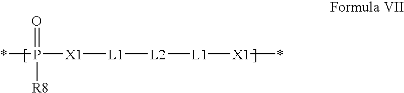
wherein, independently for each occurrence:
   L2 is a divalent organic group as described in greater detail below; and
   the other moieties are as defined as above.

In Formula VII, L2 may be a divalent, branched or straight chain aliphatic group, a cycloaliphatic group, or a group of the formula:

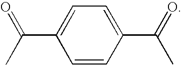

Specific examples of particular divalent, branched or straight chain aliphatic groups include an alkylene group with 1 to 7 carbon atoms, such as 2-methylpropylene or ethylene. Specific examples of cycloaliphatic groups include cycloalkylene groups, such as cyclopentylene, 2-methylcyclopentylene, cyclohexylene and 2-chloro-cyclohexylene; cycloalkenylene groups, such as cyclohexenylene; and cycloalkylene groups having fused or bridged additional ring structures, such as tetralinylene, decalinylene and norpinanylene; or the like.

In certain embodiments of the monomeric units set forth in Formula VII, in which the entire polymer may or may not be composed of such units, the following moieties for X1, L1 and R8 may be used:

| Abbreviation | All X1 | All L1 | L2 | R8 |
|---|---|---|---|---|
| P(trans-CHDM/HOP) | O | —CH2— | 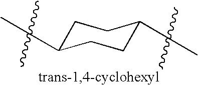 | —O(CH2)5CH3 |
| P(cis- and trans-CHDM/HOP) | O | —CH2— | 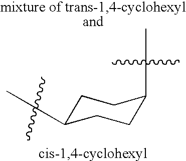 | —O(CH2)5CH3 |
| P(trans-CHDM/BOP) | O | —CH2— | trans-1,4-cyclohexyl | —O(CH2)3CH3 |
| P(trans-CHDM/EOP) | O | —CH2— | trans-1,4-cyclohexyl | —OCH2CH3 |

In another embodiment of the present invention, the polymeric compositions of the present invention include one or more recurring monomeric units represented in general Formula VIII: 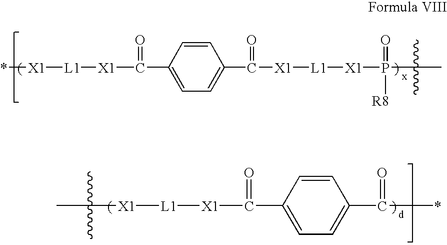
wherein, independently for each occurrence, d is equal to one or more, and optionally two, x is equal to or greater than one, and all of the other moieties are as defined above. In certain embodiments of Formula VIII, each of L1 independently may be an alkylene group, a cycloaliphatic group, a phenylene group or a divalent group of the formula: 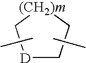
   wherein D is O, N or S and m is 0 to 3. Alternatively, L1 is a branched or straight chain alkylyene group having from 1 to 7 carbon atoms, such as a methylene, ethylene, n-propylene, 2-methylpropylene, 2,2′-dimethylpropylene group and the like.

In certain embodiments of the monomeric units set forth in Formula VIII, in which the entire polymer may or may not be composed of such units, the following moieties for X1, L1 and R8 may be used (with a variety of different x possible for each example, and, in some embodiments, d equal to two):

| | All | | |
|---|---|---|---|
| Abbreviation | X1 | All L1 | R8 |
| P(BHET-EOP/TC) | O | —CH2CH2- | —OCH2CH3 |
| P(BHDPT-EOP/TC) | O | —CH2CH(CH3)2CH2- | —OCH2CH3 |
| P(BHDPT-HOP/TC) | O | —CH2CH(CH3)2CH2- | —OC6H13 |
| P(BHPT-EOP/TC) | O | —CH2CH2CH2- | —OCH2CH3 |
| P(BHMPT-EOP/TC) | O | CH2CH2(CH3)CH2- | —OCH2CH3 |

In Formula VIII, the aryl groups represented therein may be substituted with a non-interfering substituent, for example, a hydroxy-, halogen-, or nitrogen-substituted moiety.

Other phosphorus containing polymers which may be adapted for use in the subject invention, and methods of making the same, are described in the art, including those described in U.S. Pat. Nos. 6,403,675, 6,322,797, 6,166,173, 6,376,644,5,256,765 and 5,194,581; and PCT publications WO 98/44020, WO 98/44021, and WO 98/48859. For all of the above-identified groups, non-interfering substituents may also be present.

In certain embodiments, the polymers are comprised almost entirely, if not entirely, of the same subunit. Alternatively, in other embodiments, the polymers may be copolymers, in which different subunits and/or other monomeric units are incorporated into the polymer. In certain instances, the polymers are random copolymers, in which the different subunits and/or other monomeric units are distributed randomly throughout the polymer chain. For example, the polymer having units of Formula V may consist of effectively only one type of such subunit, or alternatively two or more types of such subunits. In addition, the polymer may contain monomeric units other than those subunits represented by Formula V.

In other embodiments, the different types of monomeric units, be they one or more subunits depicted by the subject formulas or other monomeric units, are distributed randomly throughout the chain. In part, the term “random” is intended to refer to the situation in which the particular distribution or incorporation of monomeric units in a polymer that has more than one type of monomeric units is not directed or controlled directly by the synthetic protocol, but instead results from features inherent to the polymer system, such as the reactivity, amounts of subunits and other characteristics of the synthetic reaction or other methods of manufacture, processing or treatment.

In certain embodiments, the subject polymers may be cross-linked. For example, substituents of the polymeric chain, may be selected to permit additional inter-chain cross-linking by covalent or electrostatic (including hydrogen-binding or the formation of salt bridges), e.g., by the use of a organic residue appropriately substituted.

The ratio of different subunits in any polymer as described above may vary. For example, in certain embodiments, polymers may be composed almost entirely, if not entirely, of a single monomeric element, such as a subunit depicted in Formula V. Alternatively, in other instances, the polymers are effectively composed of two different subunits, in which the percentage of each subunit may vary from less than 1:99 to more than 99:1, or alternatively 10:90, 15:85, 25:75, 40:60, 50:50, 60:40, 75:25, 85:15, 90:10 or the like. For example, in some instances, a polymer may be composed of two different subunits that may be both represented by the generic Formula V, but which differ in their chemical identity. In certain embodiments, the polymers may have just a few percent, or even less (for example, about 5, 2.5, 1, 0.5, 0.1%) of the subunits having phosphorous-based linkages. In other embodiments, in which three or more different monomeric units are present, the present invention contemplates a range of mixtures like those taught for the two-component systems.

In certain embodiments, the polymeric chains of the subject compositions, e.g., which include repetitive elements shown in any of the subject formulas, have molecular weights ranging from about 2000 or less to about 1,000,000 or more daltons, or alternatively about 10,000, 20,000, 30,000, 40,000, or 50,000 daltons, more particularly at least about 100,000 daltons, and even more specifically at least about 250,000 daltons or even at least 500,000 daltons. Number-average molecular weight (Mn) may also vary widely, but generally fall in the range of about 1,000 to about 200,000 daltons, or even from about 1,000 to about 100,000 daltons, or even from about 1,000 to about 50,000 daltons. In some embodiments, Mn varies between about 8,000 and 45,000 daltons. Within a given sample of a subject polymer, a wide range of molecular weights may be present. For example, molecules within the sample may have molecular weights which differ by a factor of 2, 5, 10, 20, 50, 100, or more, or which differ from the average molecular weight by a factor of 2, 5, 10, 20, 50, 100, or more.

One method to determine molecular weight is by gel permeation chromatography (“GPC”), e.g., mixed bed columns, CH₂Cl₂ solvent, light scattering detector, and off-line dn/dc. Other methods are known in the art.

In certain embodiments, the intrinsic viscosities of the polymers generally vary from about 0.01 to about 2.0 dL/g in chloroform at 40° C., alternatively from about 0.01 to about 1.0 dL/g and, occasionally, from about 0.01 to about 0.5 dL/g.

The glass transition temperature (Tg) of the subject polymers may vary widely, and depend on a variety of factors, such as the degree of branching in the polymer components, the relative proportion of phosphorous-containing monomer used to make the polymer, and the like. When the article of the invention is a rigid solid, the Tg is often within the range of from about −10° C. to about 80° C., particularly between about 0 and 50° C. and, even more particularly between about 25° C. to about 35° C. In other embodiments, the Tg may be low enough to keep the composition of the invention flowable at body temperature. Then, the glass transition temperature of the polymer used in the invention is usually about 0 to about 37° C., or alternatively from about 0 to about 25° C.

In certain embodiments, substituents of the phosphorus atom, such as R8 in the above formulas, and other components of the subject polymers may permit additional inter-chain cross-linking by covalent or electrostatic interactions (including, for example, hydrogen-binding or the formation of salt bridges) by having a side chain of either of them appropriately substituted as discussed in greater detail below.

In other embodiments, the polymer composition of the invention may be a flexible or flowable material. When the polymer used is itself flowable, the polymer composition of the invention, even when viscous, need not include a biocompatible solvent to be flowable, although trace or residual amounts of biocompatible solvents may still be present.

In certain embodiments, a fluid polymer may be especially suitable for the treatment of CNS neoplasms. A fluid material may be adapted for injection or instillation into a tissue mass or into an actual or potential space. Certain types of fluid polymers may be termed flowable. A flowable material, often capable of assuming the shape of the contours of an irregular space, may be delivered to a portion of an actual or potential space to flow therefrom into a larger portion of the space. In this way, the flowable material may come to coat an entire post-operative surgical site after being inserted through an edge of an incision or after being instilled through a drain or catheter left in the surgical bed. Alternatively, if the flowable material is inserted under pressure through a device such as a needle or a catheter, it may perform hydrodissection, thus opening up a potential space and simultaneously coating the space with polymer. Such potential spaces suitable for hydrodissection may be found in various identifiable anatomic areas in the brain, spinal cord, cranium, and associated regions. A flowable polymer may be particularly adapted for instillation through a needle, catheter or other delivery device such as an endoscope, since its flowable characteristics allow it to reach surfaces that extend beyond the immediate reach of the delivery device. A flowable polymer in a highly fluid state may be suitable for injection through needles or catheters into tissue masses, such as tumors or margins of resection sites. Physical properties of polymers may be adjusted to achieve a desirable state of fluidity or flowability by modification of their chemical components and crosslinking, using methods familiar to practitioners of ordinary skill in the art.

A flexible polymer may be used in the fabrication of a solid article. Flexibility involves having the capacity to be repeatedly bent and restored to its original shape. Solid articles made from flexible polymers are adapted for placement in anatomic areas where they will encounter the motion of adjacent organs or body walls. Certain areas of motion are familiar to practitioners dealing with CNS neoplasms. A flexible solid article can thus be sufficiently deformed by those moving tissues that it does not cause tissue damage. Flexibility is particularly advantageous where a solid article might be dislodged from its original position and thereby encounter an unanticipated moving structure; flexibility may allow the solid article to bend out of the way of the moving structure instead of injuring it. Such a flexible article might be suitable for covering pulsatile vessels such as the internal carotid artery, the cerebral arteries, the middle meningeal artery, the basilar artery, the vertebral artery, and the spinal arteries, or for covering more delicate structures in the head such as the venous sinuses that may also be affected by local movements. Similarly, a flexible solid article may be used to protect nerves exposed during a cranial or spinal dissection such as the spinal accessory nerve, wherein the flexibility of the solid article may permit it to bend or deform when encountering motion rather than eroding into or damaging the nerve. Use of a solid article according to the present invention in the aforesaid ways may allow less extensive dissections to be carried out with surgical preservation and protection of structures important to function. Solid articles may be configured as three-dimensional structures suitable for implantation in specific anatomic areas. For example, a solid article implantable into the margins of a resected bone or cartilaginous structure may be fabricated according to the present invention to carry an antineoplastic taxane. Solid articles may be formed as films, meshes, sheets, tubes, or any other shape appropriate to the dimensions and functional requirements of the particular anatomic area. Physical properties of polymers may be adjusted to attain a desirable degree of flexibility by modification of the chemical components and crosslinking thereof, using methods familiar to practitioners of ordinary skill in the art.

While it is possible that the biocompatible polymer or the biologically active agent may be dissolved in a small quantity of a solvent that is non-toxic to more efficiently produce an amorphous, monolithic distribution or a fine dispersion of the biologically active agent in the flexible or flowable composition, it is an advantage of the invention that, in some embodiments, no solvent is needed to form a flowable composition. Moreover, the use of solvents may be avoided because, once a polymer composition containing solvent is placed totally or partially within the body, the solvent dissipates or diffuses away from the polymer and must be processed and eliminated by the body, placing an extra burden on the body's clearance ability at a time when the illness (and/or other treatments for the illness) may have already deleteriously affected it.

However, when a solvent is used to facilitate mixing or to maintain the flowability of the polymer composition of the invention, it should be non-toxic, otherwise biocompatible, and should be used in relatively small amounts. Solvents that are toxic clearly should not be used in any material to be placed even partially within a living body. Such a solvent also must not cause substantial tissue irritation or necrosis at the site of administration.

Examples of suitable biocompatible solvents, when used, include N-methyl-2-pyrrolidone, 2-pyrrolidone, ethanol, propylene glycol, acetone, methyl acetate, ethyl acetate, methyl ethyl ketone, dimethylformamide, dimethyl sulfoxide, tetrahydrofuran, caprolactam, dimethyl-sulfoxide, oleic acid, or 1-dodecylazacycloheptan-2-one. Solvents may include N-methyl-2-pyrrolidone, 2-pyrrolidone, dimethyl sulfoxide, and acetone because of their solvating ability and their biocompatibility.

The microspheres may be manufactured by incorporating the drug into the polymer matrix by either dissolving or suspending the drug into polymer solution and the mixture will be subsequently dried by techniques familiar to those skill in the arts to form microspheres. These techniques include but not limited to spray drying, coating, various emulsion methods and supercritical fluid processing. The microspheres may be mixed with a pharmaceutically acceptable diluent prior to the administration for injection. They may also be directly applied to the desired site, such as a surgical wound or cavity, by various delivery systems including pouring and spraying. The microspheres may also be mixed with pharmaceutically acceptable ingredients to create ointment or cream for topical applications.

In certain embodiments, the subject polymers are soluble in one or more common organic solvents for ease of fabrication and processing. Common organic solvents include such solvents as chloroform, dichloromethane, dichloroethane, 2-butanone, butyl acetate, ethyl butyrate, acetone, ethyl acetate, dimethyl

Therapeutic Compositions

The antineoplastic agents of the present invention are used in amounts that are therapeutically effective, which varies widely depending largely on the particular antineoplastic agent being used and the CNS being treated. The amount of antineoplastic agent incorporated into the composition also depends upon the desired release profile, the concentration of the agent required for a biological effect, and the length of time that the biologically active substance has to be released for treatment. In certain embodiments, the biologically active substance may be blended with the polymer matrix of the invention at different loading levels, in some embodiments, at room temperature and without the need for an organic solvent. In other embodiments, the compositions of the present invention may be formulated as microspheres.

There is no critical upper limit on the amount of antineoplastic agent incorporated except for that of an acceptable solution or dispersion viscosity to maintain the physical characteristics desired for the composition. The lower limit of the antineoplastic agent incorporated into the polymer system is dependent upon the activity of the drug and the length of time needed for treatment. Thus, the amount of the antineoplastic agent should not be so small that it fails to produce the desired physiological effect, nor so large that the antineoplastic agent is released in an uncontrollable manner. Typically, within these limits, amounts of the antineoplastic agent from about 1% up to about 60% may be incorporated into the present delivery systems. However, lesser amounts may be used to achieve efficacious levels of treatment for antineoplastic agents that are particularly potent.

In addition, the polymer compositions of the invention may comprise blends of the polymer of the invention with other biocompatible polymers or copolymers, so long as the additional polymers or copolymers do not interfere undesirably with the biocompatible, biodegradable and/or mechanical characteristics of the composition. Blends of the polymer of the invention with such other polymers may offer even greater flexibility in designing the precise release profile desired for targeted drug delivery or the precise rate of biodegradability desired. Examples of such additional biocompatible polymers include other poly(phosphoesters), poly(carbonates), poly(esters), poly(orthoesters), poly(amides), poly(urethanes), poly(imino-carbonates), and poly(anhydrides).

Pharmaceutically acceptable polymeric carriers may also comprise a wide range of additional materials. Without being limited thereto, such materials may include diluents, binders and adhesives, lubricants, disintegrants, colorants, bulking agents, flavorings, sweeteners, and miscellaneous materials such as buffers and adsorbents, in order to prepare a particular medicated composition, with the condition that none of these additional materials will interfere with the intended purpose of the subject composition.

For delivery of an antineoplastic agent or some other biologically active substance, the agent or substance is added to the polymer composition. A variety of methods are known in the art for encapsulating a biologically active substance in a polymer. For example, the agent or substance may be dissolved to form a homogeneous solution of reasonably constant concentration in the polymer composition, or it may be dispersed to form a suspension or dispersion within the polymer composition at a desired level of “loading” (grams of biologically active substance per grams of total composition including the biologically active substance, usually expressed as a percentage).

In part, a polymer composition of the present invention useful in the treatment of CNS neoplasms includes both: (a) an antineoplastic agent, and (b) a biocompatible and optionally biodegradable polymer, such as one having the recurring monomeric units shown in one of the foregoing formulas, or any other biocompatible polymer mentioned above or known in the art. In certain embodiments in which the subject composition will be used to treat a CNS neoplasm, the antineoplastic agent is an antineoplastic taxane, such as paclitaxel, docetaxel, an analog thereof, or another antineoplastic taxane. In still other embodiments, the subject compositions encapsulate more than one antineoplastic agent for treatment of one or more CNS neoplasms.

In addition to an antineoplastic agent, the subject compositions may contain another therapeutic agent. Any additional therapeutic agent in a subject composition may vary widely with the purpose for the composition. The term therapeutic agent includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

Plasticizers and stabilizing agents known in the art may be incorporated in polymers of the present invention. In certain embodiments, additives such as plasticizers and stabilizing agents are selected for their biocompatibility.

A composition of this invention may further contain one or more adjuvant substances, such as fillers, thickening agents or the like. In other embodiments, materials that serve as adjuvants may be associated with the polymer matrix. Such additional materials may affect the characteristics of the polymer matrix that results. For example, fillers, such as bovine serum albumin (BSA) or mouse serum albumin (MSA), may be associated with the polymer matrix. In certain embodiments, the amount of filler may range from about 0.1 to about 50% or more by weight of the polymer matrix, or about 2.5, 5, 10, 25, 40 percent. Incorporation of such fillers may affect the biodegradation of the polymeric material and/or the sustained release rate of any encapsulated substance. Other fillers known to those of skill in the art, such as carbohydrates, sugars, starches, saccharides, celluoses and polysaccharides, including mannitose and sucrose, may be used in certain embodiments in the present invention.

In other embodiments, spheronization enhancers facilitate the production of subject polymeric matrices that are generally spherical in shape. Substances such as zein, microcrystalline cellulose or microcrystalline cellulose co-processed with sodium carboxymethyl cellulose may confer plasticity to the subject compositions as well as implant strength and integrity. In particular embodiments, during spheronization, extrudates that are rigid, but not plastic, result in the formation of dumbbell shaped implants and/or a high proportion of fines, and extrudates that are plastic, but not rigid, tend to agglomerate and form excessively large implants. In such embodiments, a balance between rigidity and plasticity is desirable. The percent of spheronization enhancer in a formulation depends on the other excipient characteristics and is typically in the range of 10-90% (w/w).

Buffers, acids and bases may be incorporated in the subject compositions to adjust their pH. Agents to increase the diffusion distance of agents released from the polymer matrix may also be included.

Disintegrants are substances which, in the presence of liquid, promote the disruption of the subject compositions. Disintegrants are most often used in implants, in which the function of the disintegrant is to counteract or neutralize the effect of any binding materials used in the subject formulation. In general, the mechanism of disintegration involves moisture absorption and swelling by an insoluble material. Examples of disintegrants include croscarmellose sodium and crospovidone that, in certain embodiments, may be incorporated into the polymeric matrices in the range of about 1-20% of total matrix weight. In other cases, soluble fillers such as sugars (mannitol and lactose) may also be added to facilitate disintegration of the subject compositions upon use.

Other materials may be used to advantage to control the desired release rate of an antineoplastic agent for a particular treatment protocol. For example, if the sustained release is too slow for a particular application, a pore-forming agent may be added to generate additional pores in the matrix. Any biocompatible water-soluble material may be used as the pore-forming agent. They may be capable of dissolving, diffusing or dispersing out of the formed polymer system whereupon pores and microporous channels are generated in the system. The amount of pore-forming agent (and size of dispersed particles of such pore-forming agent, if appropriate) within the composition should affect the size and number of the pores in the polymer system.

Pore-forming agents include any pharmaceutically acceptable organic or inorganic substance that is substantially miscible in water and body fluids and will dissipate from the forming and formed matrix into aqueous medium or body fluids or water-immiscible substances that rapidly degrade to water-soluble substances. Suitable pore-forming agents include, for example, sugars such as sucrose and dextrose, salts such as sodium chloride and sodium carbonate, and polymers such as hydroxylpropylcellulose, carboxymethylcellulose, polyethylene glycol, and polyvinylpyrrolidone. The size and extent of the pores may be varied over a wide range by changing the molecular weight and percentage of pore-forming agent incorporated into the polymer system.

The charge, lipophilicity or hydrophilicity of any subject polymeric matrix may be modified by attaching in some fashion an appropriate compound to the surface of the matrix. For example, surfactants may be used to enhance wettability of poorly soluble or hydrophobic compositions. Examples of suitable surfactants include dextran, polysorbates and sodium lauryl sulfate. In general, surfactants are used in low concentrations, generally less than about 5%.

Binders are adhesive materials that may be incorporated in polymeric formulations to bind and maintain matrix integrity. Binders may be added as dry powder or as solution. Sugars and natural and synthetic polymers may act as binders. Materials added specifically as binders are generally included in the range of about 0.5%-15% w/w of the matrix formulation. Certain materials, such as microcrystalline cellulose, also used as a spheronization enhancer, also have additional binding properties.

Various coatings may be applied to modify the properties of the matrices. Three exemplary types of coatings are seal, gloss and enteric coatings. Other types of coatings having various dissolution or erosion properties may be used to further modify subject matrices behavior, and such coatings are readily known to one of ordinary skill in the art.

The seal coat may prevent excess moisture uptake by the matrices during the application of aqueous based enteric coatings. The gloss coat generally improves the handling of the finished matrices. Water-soluble materials such as hydroxypropyl cellulose may be used to seal coat and gloss coat implants. The seal coat and gloss coat are generally sprayed onto the matrices until an increase in weight between about 0.5% and about 5%, often about 1% for a seal coat and about 3% for a gloss coat, has been obtained.

Enteric coatings consist of polymers which are insoluble in the low pH (less than 3.0) of the stomach, but are soluble in the elevated pH (greater than 4.0) of the small intestine. Polymers such as EUDRAGIT, RohmTech, Inc., Malden, Mass., and AQUATERIC, FMC Corp., Philadelphia, Pa., may be used and are layered as thin membranes onto the implants from aqueous solution or suspension or by a spray drying method. The enteric coat is generally sprayed to a weight increase of about one to about 30%, or about 10 to about 15% and may contain coating adjuvants such as plasticizers, surfactants, separating agents that reduce the tackiness of the implants during coating, and coating permeability adjusters.

The present compositions may additionally contain one or more optional additives such as fibrous reinforcement, colorants, perfumes, rubber modifiers, modifying agents, etc. In practice, each of these optional additives should be compatible with the resulting polymer and its intended use. Examples of suitable fibrous reinforcement include PGA microfibrils, collagen microfibrils, cellulosic microfibrils, and olefinic microfibrils. The amount of each of these optional additives employed in the composition is an amount necessary to achieve the desired effect.

Physical structures of the subject compositions The subject polymers may be formed in a variety of shapes. For example, in certain embodiments, subject polymer matrices may be presented in the form of microparticles or nanoparticles. Such particles may be prepared by a variety of methods known in the art, including for example, solvent evaporation, spray-drying or double emulsion methods.

The shape of microparticles and nanoparticles may be determined by scanning electron microscopy. Spherically shaped nanoparticles are used in certain embodiments for circulation through the bloodstream. If desired, the particles may be fabricated using known techniques into other shapes that are more useful for a specific application.

In addition to intracellular delivery of an antineoplastic taxane, it also possible that particles of the subject compositions, such as microparticles or nanoparticles, may undergo endocytosis, thereby obtaining access to the cell. The frequency of such an endocytosis process will likely depend on the size of any particle.

In certain embodiments, solid articles useful in defining shape and providing rigidity and structural strength to the polymeric matrices may be used. For example, a polymer may be formed on a mesh or other weave for implantation. A polymer may also be fabricated as a stent or as a shunt, adapted for holding open areas within body tissues or for draining fluid from one body cavity or body lumen into another. Further, a polymer may be fabricated as a drain or a tube suitable for removing fluid from a post-operative site, and in some embodiments adaptable for use with closed section drainage systems such as Jackson-Pratt drains and the like familiar in the art.

The mechanical properties of the polymer may be important for the processability of making molded or pressed articles for implantation. For example, the glass transition temperature may vary widely but must be sufficiently lower than the temperature of decomposition to accommodate conventional fabrication techniques, such as compression molding, extrusion or injection molding.

Biodegradability and Release Characteristics

In certain embodiments, the polymers and blends of the present invention, upon contact with body fluids, undergo gradual degradation. The life of a biodegradable polymer in vivo depends, among other things, upon its molecular weight, crystallinity, biostability, and the degree of crosslinking. In general, the greater the molecular weight, the higher the degree of crystallinity, and the greater the biostability, the slower biodegradation will be.

If a subject polymer matrix is formulated with an antineoplastic agent, release of such agent for a sustained or extended period as compared to the release from an isotonic saline solution generally results. Such release profile may result in prolonged delivery (over, say 1 to about 4,000 hours, or alternatively about 4 to about 1500 hours) of effective amounts (e.g., about 0.00001 mg/kg/hour to about 10 mg/kg/hour) of the agent associated with the polymer.

A variety of factors may affect the desired rate of hydrolysis of polymers of the subject invention, the desired softness and flexibility of the resulting solid matrix, rate and extent of bioactive material release. Some of such factors include: the selection of the various substituent groups, such as the phosphate group making up the linkage in the polymer backbone (or analogs thereof), the enantiomeric or diastereomeric purity of the monomeric subunits, homogeneity of subunits found in the polymer, and the length of the polymer. For instance, the present invention contemplates heteropolymers with varying linkages, and/or the inclusion of other monomeric elements in the polymer, in order to control, for example, the rate of biodegradation of the matrix.

To illustrate further, a wide range of degradation rates may be obtained by adjusting the hydrophobicities of the backbones or side chains of the polymers while still maintaining sufficient biodegradability for the use intended for any such polymer. Such a result may be achieved by varying the various functional groups of the polymer. For example, the combination of a hydrophobic backbone and a hydrophilic linkage produces heterogeneous degradation because cleavage is encouraged whereas water penetration is resisted. In another example, it is expected that use of substituent on phosphate in the polymers of the present invention that is lipophilic, hydrophobic or bulky group would slow the rate of degradation. For example, it is expected that conversion of the phosphate side chain to a more lipophilic, more hydrophobic or more sterically bulky group would slow down the rate of biodegradation. Thus, release is usually faster from polymer compositions with a small aliphatic group side chain than with a bulky aromatic side chain.

One protocol generally accepted in the field that may be used to determine the release rate of any antineoplastic agent or other material loaded in the polymer matrices of the present invention involves degradation of any such matrix in a 0.1 M PBS solution (pH 7.4) at 37° C., an assay known in the art For purposes of the present invention, the term “PBS protocol” is used herein to refer to such protocol.

In certain instances, the release rates of different polymer systems of the present invention may be compared by subjecting them to such a protocol. In certain instances, it may be necessary to process polymeric systems in the same fashion to allow direct and relatively accurate comparisons of different systems to be made. For example, the present invention teaches several different means of formulating the polymeric matrices of the present invention. Such comparisons may indicate that any one polymeric system releases incorporated material at a rate from about 2 or less to about 1000 or more times faster than another polymeric system. Alternatively, a comparison may reveal a rate difference of about 3, 5, 7, 10, 25, 50, 100, 250, 500 or 750. Even higher rate differences are contemplated by the present invention and release rate protocols.

In certain embodiments, when formulated in a certain manner, the release rate for polymer systems of the present invention may present as mono- or bi-phasic. Release of any material incorporated into the polymer matrix, which is often provided as a microsphere, may be characterized in certain instances by an initial increased release rate, which may release from about 5 to about 50% or more of any incorporated material, or alternatively about 10, 15, 20, 25, 30 or 40%, followed by a release rate of lesser magnitude.

The release rate of any incorporated material may also be characterized by the amount of such material released per day per mg of polymer matrix. For example, in certain embodiments, the release rate may vary from about 1 ng or less of any incorporated material per day per mg of polymeric system to about 5000 or more ng/day.mg. Alternatively, the release rate may be about 10, 25, 50, 75, 100, 125, 150, 175, 200, 250, 300, 350, 400, 450, 500, 600, 700, 800 or 900 ng/day.mg. In still other embodiments, the release rate of any incorporated material may be 10,000 ng/day.mg or even higher. In certain instances, materials incorporated and characterized by such release rate protocols may include antineoplastic agents, fillers, and other substances.

In another aspect, the rate of release of any material from any polymer matrix of the present invention may be presented as the half-life of such material in the such matrix.

In addition to the embodiment involving protocols for in vitro determination of release rates, in vivo protocols, whereby in certain instances release rates for polymeric systems may be determined in vivo, are also contemplated by the present invention. Other assays useful for determining the release of any material from the polymers of the present system are known in the art.

4. Implants and Delivery Systems

In its simplest form, a delivery system for an antineoplatic agent for treatment of a CNS neoplasm consists of a dispersion of such an agent into one of the polymers described above. In other embodiments, an article is used for implantation, injection, or otherwise placed totally or partially within an anatomic area, the article comprising a subject composition for treatment of a CNS neoplasm. It may be particularly important that such an article result in minimal tissue irritation when applied to, implanted in or injected into vascularized tissue, hypovascularized post-operative tissue or tissue exposed to previous radiation that is part of the CNS. In certain embodiments, a solid, flowable or fluid article comprising the composition of the invention is inserted within an anatomic area by implantation, injection, endoscopy or otherwise being placed within an anatomic area of the subject being treated for a CNS neoplasm.

As a structural medical device, the polymer compositions of the inventions provide a wide variety of physical forms having specific chemical, physical and mechanical properties suitable for insertion into an anatomic area.

Biocompatible delivery systems and articles thereof, may be prepared in a variety of ways known in the art. The subject polymer may be melt processed using conventional extrusion or injection molding techniques, or these products may be prepared by dissolving in an appropriate solvent, followed by formation of the device, and subsequent removal of the solvent by evaporation or extraction, e.g., by spray drying. By these methods, the polymers may be formed into articles of almost any size or shape desired, for example, implantable solid discs or wafers or injectable rods, microspheres, or other microparticles.

Typical medical articles also include such as implants as laminates for degradable fabric or coatings to be placed on other implant devices.

In one embodiment, certain polymer compositions of the subject invention may be used to form a soft, drug-delivery “depot” that can be administered as a liquid, for example, by injection, but which remains sufficiently viscous to maintain the drug within the localized area around the injection site. By using a polymer composition in flowable form, even the need to make an incision can be eliminated. In any event, the flexible or flowable delivery “depot” will adjust to the shape of the space it occupies within the body with a minimum of trauma to surrounding tissues.

When the polymer composition of the invention is flexible or flowable, it may be placed in an anatomic area of the CNS. It may be inserted into the anatomic area either through an open surgical wound, under direct or indirect vision, or through any of the access devices routinely used in the art to enter such areas, for example, indwelling or acutely-inserted catheters, needles, drains, superselective angiography means and the like. A flowable or fluid polymer may be adapted for mixing with the transudate or exudate found within or expected to gather within the anatomic area. A flowable or fluid polymer may be instilled in an anatomic area during surgery on organs or structures therein to decrease the likelihood of recurrent disease when there is a high risk for its development. In certain embodiments, a polymer composition according to the present invention may also be incorporated in access devices so that an antineoplastic agent is released into the anatomic area within which the access device resides, thereby decreasing the size of a primary or recurrent CNS neoplasm, treating said neoplasm, or preventing the development of recurrent disease where a neoplasm has been extirpated. The polymer composition of the invention may also be used to produce coatings for other solid implantable devices for use in treatment of CNS neoplasms.

Once a system or implant article is in place, it should remain in at least partial contact with a biological fluid, such as blood, tissue fluid, lymph, or secretions from organ surfaces or mucous membranes, and the like to allow for sustained release of any encapsulated antineoplastic agent, e.g., an antineoplastic taxane.

5. Exemplary Methods of Making the Subject Polymers

In general, the polymers of the present invention may be prepared by melt polycondensation, solution polymerization or interfacial polycondensation. Techniques necessary to prepare the subject polymers are known in the art, and reference is made in particular to U.S. patent application Ser. No. 09/885,085, filed Jun. 21, 2001, which is hereby incorporated by this reference in its entirety.

The most common general reaction in preparing the subject compositions is a dehydrochlorination between a phosphodichloridate and a diol according to the following equation:

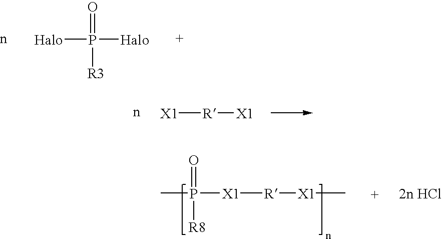

Certain of the subject polymers may be obtained by condensation between appropriately substituted dichlorides and diols.

An advantage of melt polycondensation is that it avoids the use of solvents and large amounts of other additives, thus making purification more straightforward. This method may also provide polymers of reasonably high molecular weight. Somewhat rigorous conditions, however, are often required and may lead to chain acidolysis (or hydrolysis if water is present). Unwanted, thermally induced side reactions, such as cross-linking reactions, may also occur if the polymer backbone is susceptible to hydrogen atom abstraction or oxidation with subsequent macroradical recombination.

To minimize these side reactions, the polymerization may also be carried out in solution. Solution polycondensation requires that both the prepolymer and the phosphorus component be sufficiently soluble in a common solvent. Typically, a chlorinated organic solvent is used, such as chloroform, dichloromethane or dichloroethane. The solution polymerization is generally run in the presence of equimolar amounts of the reactants and, in some embodiments, an excess of an acid acceptor and a catalyst, such as 4-dimethylaminopyridine (DMAP). Useful acid acceptors include tertiary amines as pyridine or triethylamine. The product is then typically isolated from the solution by precipitation in a non-solvent and purified to remove the hydrochloride salt by conventional techniques known to those of ordinary skill in the art, such as by washing with an aqueous acidic solution, e.g., dilute HCl.

Reaction times tend to be longer with solution polymerization than with melt polymerization. However, because overall milder reaction conditions may be used, side reactions are minimized, and more sensitive functional groups may be incorporated into the polymer. The disadvantages of solution polymerization are that removal of solvents may be difficult.

Interfacial polycondensation may be used when high molecular-weight polymers are desired at high reaction rates. By such methods, mild conditions minimize side reactions, and the dependence of high molecular weight on stoichiometric equivalence between diol and dichloridate inherent in solution methods is removed. However, hydrolysis of the acid chloride may occur in the alkaline aqueous phase, and sensitive dichloridates that have some solubility in water are generally subject to hydrolysis rather than polymerization. Phase transfer catalysts, such as crown ethers or tertiary ammonium chloride, may be used to bring the ionized diol to the interface to facilitate the polycondensation reaction. The yield and molecular weight of the resulting polymer after interfacial polycondensation are affected by reaction time, molar ratio of the monomers, volume ratio of the immiscible solvents, the type of acid acceptor, and the type and concentration of the chase transfer catalyst.

Methods for making the present invention may take place at widely varying temperatures, depending upon whether a solvent is used and, if so, which one; the molecular weight desired; the susceptibility of the reactants to form side reactions; and the presence of a catalyst. Usually, the process takes place at a temperature ranging from about 0 to about +235° C. for melt conditions. Somewhat lower temperatures, e.g., for example from about −50 to about 100° C., may be possible with solution polymerization or interfacial polycondensation with the use of either a cationic or anionic catalyst.

The time required for the process may vary widely, depending on the type of reaction being used, the molecular weight desired and, in general, the need to use more or less rigorous conditions for the reaction to proceed to the desired degree of completion. Typically, however, the synthetic process takes place during a time between about 30 minutes and about 7 days.

Although the process may be in bulk, in solution, by interfacial polycondensation, or any other convenient method of polymerization, in many instant embodiments, the process takes place under solution conditions. Particularly useful solvents include methylene chloride, chloroform, tetrahydrofuran, di-methyl formamide, dimethyl sulfoxide or any of a wide variety of inert organic solvents.

In greater detail, polymers of Formula VI may be prepared, at least in part, by reacting a compound having a formula H-Y1-L1-Y1-H, such as 2-aminoethanol, ethylene glycol, ethane dithiol, etc., with a cyclic compound, e.g., having one of the following structures: for example, caprolactone or lactide (lactic acid dimer).

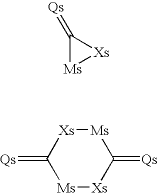

Thus, the cyclic compound may include one or two subunits ts. For cyclic compounds containing two subunits, the two subunits contained therein may be the same or different.

For synthesizing, for example, a compound of Formula VI, wherein x and y are on average about 10, an equivalent of ethylene glycol as H—Y1-L1-Y1—H may be reacted with equivalents of 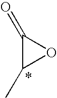
or 10 equivalents of 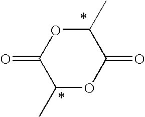
because lactic acid dimer contains two monomer units for each equivalent of the cyclic compound. Variation of the ratio of cyclic compound to ethylene glycol or other bifunctional core will likewise vary the values of x and y, although x and y will be substantially equal for a symmetrical bifunctional core (e.g., ethylene glycol) for subject polymers prepared by this method. For an unsymmetrical bifunctional core (e.g., 2-aminoethanol), the ratio of x:y may vary considerably, as will be understood by one of skill in the art and may be determined without undue experimentation.

Polymers of the present invention may generally be isolated from the reaction mixture by conventional techniques, such as by precipitating out, extraction with an immiscible solvent, evaporation, filtration, crystallization and the like. Typically, the subject polymers are both isolated and purified by quenching a solution of polymer with a non-solvent or a partial solvent, such as diethyl ether or petroleum ether.

6. Exemplary Methods for Treating CNS Neoplasms

A contemplated by the present invention, the antineoplastic agent for treatment of a CNS neoplasm will be released from a subject polymer system in an amount sufficient to deliver to a patient a therapeutically effective amount of such agent as part of a prophylactic or therapeutic treatment. The desired concentration of active compound in the polymer system will depend on absorption, inactivation, and excretion rates of the drug as well as the delivery rate of the compound from the subject composition. It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions. Typically, dosing will be determined using techniques known to one skilled in the art. As one non-limiting example, dosage may be based on the amount of the antineoplastic agent encapsulated in the subject polymers. For example, a range of amounts of antineoplastic agent are contemplated, including 0.5, 1, 2, 3, 4, 5, 7.5, 10, 15, 20, 25 mg or more of such agent per kg body weight of the patient. Other amounts will be known to those of skill in the art and readily determined.

Methods for treating CNS neoplasms according to the present invention involve gaining access to an anatomic area where a disease to be treated is located or may grow and instilling therein a composition comprising a biocompatible and optionally biodegradable polymer and an antineoplastic agent. In certain embodiments, the antineoplastic agent is an antineoplastic taxane. According to the present invention, in certain embodiments the polymer composition may be a fluid, a flowable material or a rigid or flexible solid article. Access to the anatomic area is gained by techniques familiar to practitioners in the medical arts. In certain embodiments, the compositions of the present invention are instilled into the anatomic area to prevent or to minimize the occurrence or recurrence of a neoplasm in a patient who is at increased risk for developing such a disease. Optionally, the polymeric composition of the present composition may be removed at a preselected time interval after it is instilled, although certain compositions according to the present invention are formulated to reside within the body cavity for prolonged periods of time or permanently, in certain cases degrading over time or being resorbed by, digested by or metabolized by the local body tissues. Repeated instillations of the subject polymeric compositions may be undertaken, but certain compositions are formulated for sustained or extended release of the therapeutically effective amount of an antineoplastic agent, so that a single applied dose may be sufficient to treat the CNS neoplasm adequately. Combination therapies for CNS neoplasms also fall within the scope of the present invention where one component of the combination therapy involves the instillation of the compositions of the present invention as claimed and as described herein. As an example, combined treatment regimens for CNS neoplasms may involve the instillation of an antineoplastic agent within an anatomic area accompanied by another type of treatment, such as systemic chemotherapy administration or locoregional radiation therapy, thermotherapy or other therapeutic application of electromagnetic energy. Other therapeutic combinations, all falling similarly within the scope of the present invention, will be apparent to practitioners of ordinary skill in the art using no more than routine experimentation. For example, and without limitation, the modalities of the therapeutic combination may have an affect on result of treatment, such as the timing of radiation treatment.

Certain exemplary treatment methods for various aspects of CNS neoplasms are described below. It is understood, however, that these descriptions are intended as illustrative only, not intended to be limiting in any way, and that other modifications and variations of these illustrative embodiments may be contemplated without departing from the scope of the present invention.

Instillation of compositions according to these inventive methods may accompany procedures for resecting CNS neoplasms or other surgical procedures. Furthermore, these methods are consistent with prophylactic application, in those cases where the risk of developing primary or recurrent CNS neoplasms is high. For example, in a surgical procedure where extensive disease is apparent, the clinician might deem it advisable to apply the compositions of the present invention around the excisional area using any of the delivery systems that would be familiar in the art. A liquid, gel, spray, aerosol or formed article could be used under these circumstances to deploy the inventive compositions for the prevention or the minimization of recurrent disease in the future. A delivery system adapted to any of these treatments for CNS neoplasms could be fabricated and composed to carry out other desirable medical functions without exceeding the scope of the present invention: for example, an antineoplastic taxane composition according to the present invention could be combined with other substances such as anti-adhesion substances, hemostatic substances, immunogenic substances, or any other therapeutic agent without limitation.

The efficacy of treatment with the subject compositions may be determined in a number of fashions. In one method, the median survival rate or median survival time or life span for treatment with a subject composition may be compared to other forms of treatment with the same antineoplastic agent. The increase in median survival rate or time or life span for treatment with a subject composition as compared to treatment with another method may be 10, 25, 50, 75, 100, 150, 200, 300, 400% or even more. The period of time for observing any such increase may be about fifteen days, three months, six months, one year, three years, or five or more years. The comparison may be made against treatment with the antineoplastic agent itself, or administration of the agent in a pharmaceutically acceptable carrier, or administration as part of a different drug delivery device than a subject composition. The comparison may be made against the same or a different effective dosage of the antineoplastic agent. The different regiments compared may use electromagnetic radiation.

Alternatively, the different treatment regimens described above may be compared by comparing tumor volume doubling times, with the length of time required for tumor volume to double being approximately two-thirds, one-half, one-third, one-quarter, one-fifth, one-tenth, one-twentieth or even less for treatment with a subject composition as compared to treatment with another method using the same antineoplastic agent.

Alternatively, a comparison of the different treatment regimens described above may be based on the effectiveness of the treatment, with treatment with a subject composition being substantially better, or 50%, 100%, 150%, 200%, 300% more effective, than by another method using the same antineoplastic agent.

Alternatively, the different treatment regimens may be analyzed by comparing the therapeutic index for each of them, with treatment with a subject composition as compared to another regimen having a therapeutic index two, three, five or seven times that of, or even one, two, three or more orders of magnitude greater than, treatment with another method using the same antineoplastic agent.

Alternatively, the different treatment regimens may be analyzed by comparing the frequency of hypersensitivity reactions to each of them, with treatment with a subject composition reducing the number of hypersensitivity reactions by at least about 10, 25, 50, 75, 100, 150, 200 or even more percent as compared to another method using the same antineoplastic agent. Such comparisons may take into account whether the hypersensitivity reaction is significant and whether premedication is used.

7. Exemplification

The invention having been generally described, it will be more readily understood by reference to the following examples which are included merely for purposes of illustration of certain aspects and embodiments of the present invention and are not intended to limit the invention in any way.

### EXAMPLE 1

### First Synthesis of D,L-PL(PG)EOP

All glassware was dried for a minimum of 2 hours at 105° C. and allowed to cool in a desiccator or cooled under a stream of argon gas. A 28.5 g portion of D,L-lactide and 1.5 g of 1,2-propanediol (PG), obtained from Aldrich, Catalog No. 39,803, 99.5+%, in a molar ratio of 10:1, were weighed into a 250 mL 3-neck round-bottom flask. The flask was equipped with a gas joint and a stirrer bearing/shaft/paddle assembly. The mixture was evacuated and pressurized with argon five times to remove residual air and moisture. The reaction apparatus was immersed in a preheated oil bath at 135° C., connected to an argon source with an oil bubbler, and stirred at a moderate speed until all of the solid monomer had melted.

At this time, a volume of stock stannous octoate solution (about 130 mg/ml in toluene of chloroform) equivalent to 3.6 mg tin (120 ppm stannous octoate or equivalent to 35 ppm tin based upon weight of the prepolymer) was added to the melt using a 50 μL syringe. The reaction mixture was allowed to stir under a slight argon pressure for approximately 16 hours. The oil bath temperature was then reduced to about 110° C. and the residual monomer was removed under vacuum. The upper parts of the reaction assembly were heated gently with a heat gun to aid in the monomer removal. The total time under vacuum was 2-3 hours. A reflux condenser was then inserted between the gas joint and the flask in the prepolymer apparatus described above. The molten prepolymer was dissolved by adding 100 mL of chloroform to the reaction flask with stirring.

Next, 6.9 mL of triethylamine (TEA) and 1.21 g of DMAP were added to the stirring reaction mixture. The reaction mixture was then chilled to about 4° C. in an ice bath.

A solution of approximately 2.5 mL of freshly distilled ethyl dichlorophosphate (EOPCl₂) in 25 mL of chloroform was prepared in a dropping funnel. The solution in the funnel was added drop wise to the reaction mixture over a period of about 30 minutes. After the addition was complete the reaction mixture was allowed to continue stirring at about 4° C. for 10 minutes and then the ice bath was removed. The reaction mixture was allowed to warm to room temperature over about 1 hour. At this time a significant increase in viscosity of the clear solution was observed. The reaction mixture was then heated to reflux using an oil bath. Over the next hour the solution became cloudy. The reaction mixture was allowed to reflux over two nights, about 38 hours total.

At this time, a Barret trap was inserted between the condenser and the flask and 88 mL of solvent (⅔ of the total volume) were distilled from the reaction mixture. The Barret trap was removed and the reaction mixture was allowed to reflux for an additional 16 hours with the oil bath temperature between 98-102° C. Next, the oil bath temperature was increased to 115° C. for 2 hours. After this time, the reaction mixture was allowed to cool to room temperature, and 200 mL of dichloromethane was added and transferred to a separatory funnel. The reaction mixture was extracted twice with 100 mL of 0.1 M HCl and twice with 100 mL of saturated sodium chloride solution. The organic layer was isolated, dried overnight in the freezer at about −15° C. over 50 g of sodium sulfate, and filtered twice. The resulting polymer solution was poured into 1500 mL of hexane plus 500 mL of ether. The resulting mass of polymer was dried under vacuum. The Inherent Viscosity (IV) of this material was measured to be 0.39 dL/g.

### EXAMPLE 2

### Second Synthesis of D,L-PL(PG)EOP

All glassware was dried for a minimum of 2 hours at 105° C. and allowed to cool in a desiccator or cooled under a stream of argon gas. A 28.5 g portion of D,L-lactide and 1.5 g of PG (molar ratio, 10:1) were weighed into a 250 ml 3-neck round-bottom flask The flask was equipped with a gas joint and a stirrer bearing/shaft/paddle assembly. The mixture was evacuated and filled with argon five times to remove residual air and moisture. Each time the polymerization vessel was evacuated to a pressure between 0.5 and 10 Torr. The reaction apparatus was immersed in a preheated oil bath at 125° C., connected to an argon source with an oil bubbler, and stirred at a moderate speed until all of the solid monomer had melted. At this time, a volume of stock stannous octoate solution (about 130 mg/ml in toluene) equivalent to 100 ppm stannous octoate (29 ppm Sn) was added to the melt using a syringe. The reaction mixture was allowed to stir under a slight argon pressure for 3 hours.

The oil bath temperature was then reduced to about 105° C. and the residual monomer was removed under vacuum. The pressure was maintained as low as possible, typically between 0.5 and 10 Torr. The upper parts of the reaction assembly were heated gently with a heat gun to aid in the monomer removal. The total time under vacuum was 1 hour.

The prepolymer was cooled to room temperature under argon gas and allowed to stand for 12-18 hours at ambient temperature. The prepolymer was dissolved in 84 ml of chloroform with stirring and 2.5 equivalents of triethylamine (TEA) and 0.5 equivalents of DMAP were added to the stirring reaction mixture using a powder funnel. The reaction mixture was chilled to about −5 to about −15° C. in a cold bath. A solution of about 1 equivalent of distilled ethyl dichlorophosphate (EOPCl₂) in 10 ml of chloroform was prepared in a dropping funnel. The solution in the funnel was added slowly to the reaction mixture over a period of 0.5 hour.

After the addition was complete, the reaction mixture was allowed to stir at low temperature for 1 hour at −5° C. The reaction was then quenched with 1 ml of anhydrous methanol and stirred for another five minutes. Next, the reaction mixture was transferred to a 0.5 gallon vessel and mixed with 37 g of Dowex DR-2030 IER and 30 g of Dowex M43, and shaken on a mechanical shaker for 2 hour to remove residual DMAP and TEA free base and salts (the IERs had been washed with several bed volumes of methanol and chloroform and dried under vacuum at ambient temperature for about 18 hours). The resin was removed from the reaction mixture by vacuum filtration through Whatman 54 filter paper.

The resin was washed with about one bed volume of dichloromethane and the filtrate was concentrated to approximately 50 ml. The viscous filtrate was poured into 200 ml of petroleum ether to precipitate the polymer. The polymer mass was washed with 100 ml of petroleum ether and dried under vacuum. Molecular weights of the polymers were obtained from gel permeation chromatography (GPC) using both differential refractive index detection and a polystyrene calibration curve (CC) and by light scattering detection.

The molecular weight and IV data for the polymers prepared by this process are listed in the table below.

| Sample | Mw (LS), daltons | Mw (CC), daltons | IV, dL/g |
|---|---|---|---|
| 1 | 101,200 | 107,500 | 0.62 |
| 2 | 150,100 | 155,900 | 0.80 |
| 3 | 85,200 | 84,300 | — |
| 4 | 92,600 | 89,900 | — |

### EXAMPLE 3

### Synthesis of D,L-PL(EG)EOP

All glassware was dried for a minimum of 2 hours at 105° C. and allowed to cool in a desiccator or cooled under a stream of argon gas. A 100.0 g portion of D,L-lactide and 4.3 g of ethylene glycol (EG) (molar ratio, 10:1) were weighed into a 1000 ml 3-neck round-bottom flask. The flask was equipped with a gas joint and a stirrer bearing/shaft/paddle assembly. The mixture was evacuated and filled with argon five times to remove residual air and moisture. The reaction apparatus was immersed in a preheated oil bath at 135° C., connected to an argon source with an oil bubbler, and stirred at a moderate speed until all of the solid monomer had melted.

At this time, a volume of stock stannous octoate solution (about 130 mg/ml in toluene) equivalent to 120 ppm stannous octoate or 35 ppm Sn was added to the melt using a syringe. The reaction mixture was allowed to stir under a slight argon pressure for approximately 16 hours. The oil bath temperature was then reduced to about 110° C. and the residual monomer was removed under vacuum. The upper parts of the reaction assembly were heated gently with a heat gun to aid in the monomer removal. The total time under vacuum was 2-3 hours.

The molten prepolymer was dissolved in 350 ml of chloroform with stirring and 2.5 equivalents of TEA and 0.5 equivalents of DMAP were added to the stirring reaction mixture using a powder funnel. The reaction mixture was chilled to about −5° C. in a cold bath. A solution of about 1 equivalent of distilled ethyl dichlorophosphate (EOPCI₂) in 97 ml of chloroform was prepared in a dropping funnel. The solution in the funnel was added slowly to the reaction mixture over a period of 2 hours. After the addition was complete, the reaction mixture was allowed to stir at low temperature for 45 minutes at −5° C. After 2 hours a significant increase in viscosity of the clear solution was observed. The reaction was then quenched with 6.8 ml of anhydrous methanol and stirred for another five minutes.

Next, the reaction mixture was transferred to a 0.5 gallon vessel and mixed with 87 g of Dowex HCR-S IER and 104 g of Dowex-43, and shaken on a mechanical shaker for 1 hour to remove residual DMAP and TEA free base and salts (the IERs had been washed with several bed volumes of methanol and dried under vacuum at ambient temperature for about 18 hours). The resin was removed from the reaction mixture by vacuum filtration through Whatman 54 filter paper. The resin was washed with about one bed volume of dichloromethane and the filtrate was concentrated to approximately 150 ml. The viscous filtrate was poured into 2000 ml of hexane to precipitate the polymer. The polymer mass was washed with 2×200 ml of hexane and dried under vacuum. The molecular weights were determined by GPC were 40,400 for Mw (LS) and 42,000 for Mw (CC).

### EXAMPLE 4

### Synthesis of D,L-PL(HD)EOP

All glassware was dried for a minimum of 2 hours at 105° C. and allowed to cool in a desiccator or cooled under a stream of argon gas. A 100.0 g portion of D,L-lactide and 8.2 g of 1,6-hexane diol (HD) (molar ratio, 10:1) were weighed into a 1000 ml 3-neck round-bottom flask. The flask was equipped with a gas joint and a stirrer bearing/shaft/paddle assembly. The mixture was evacuated and filled with argon five times to remove residual air and moisture. The reaction apparatus was immersed in a preheated oil bath at 135° C., connected to an argon source with an oil bubbler, and stirred at a moderate speed until all of the solid monomer had melted.

At this time, a volume of stock stannous octoate solution equivalent (about 130 mg/ml in toluene) to 120 ppm stannous octoate or 35 ppm Sn was added to the melt using a syringe. The reaction mixture was allowed to stir under a slight argon pressure for approximately 16 hours. The oil bath temperature was then reduced to about 110° C. and the residual monomer was removed under vacuum. The upper parts of the reaction assembly were heated gently with a heat gun to aid in the monomer removal. The total time under vacuum was 2-3 hours.

The molten prepolymer was dissolved in 350 ml of chloroform with stirring and 2.5 equivalents of triethylamine (TEA) and 0.5 equivalents of DMAP were added to the stirring reaction mixture using a powder funnel. The reaction mixture was chilled to about −5° C. in a cold bath. A solution of about 1 equivalent of distilled ethyl dichlorophosphate (EOPCl₂) in 97 ml of chloroform was prepared in a dropping funnel. The solution in the funnel was added slowly to the reaction mixture over a period of 2 hours. After the addition was complete, the reaction mixture was allowed to stir at low temperature for 45 minutes at −5° C. After 2 hours, a significant increase in viscosity of the clear solution was observed. The reaction was then quenched with 6.8 ml of anhydrous methanol and stirred for another five minutes.

Next, the reaction mixture was transferred to a 0.5 gallon vessel and mixed with 87 g of Dowex HCR-S IER and 104 g of Dowex-43, and shaken on a mechanical shaker for 1 hour to remove residual DMAP and TEA free base and salts (the IERs had been washed with several bed volumes of methanol and dried under vacuum at ambient temperature for about 18 hours). The resin was removed from the reaction mixture by vacuum filtration through Whatman 54 filter paper. The resin was washed with about one bed volume of dichloromethane and the filtrate was concentrated to approximately 150 ml. The viscous filtrate was poured into 2000 ml of hexane to precipitate the polymer. The polymer mass was washed with 2×200 ml of hexane and dried under vacuum. The molecular weights were determined by GPC were 36,700 for Mw (LS) and 34,100 for Mw (CC). The value for IV was 0.33 dL/g.

### EXAMPLE 5

### Polymer of PG, D,L-Lactide, Glycolide, and Ethyl Dichlorophogphate

All glassware was dried for a minimum of 2 hours at 105° C. and allowed to cool in a desiccator or cooled under a stream of argon gas. A 28.5 g portion of D,L-lactide and 1.5 g of PG (molar ratio, 10:1) were weighed into a 250 ml 3-neck round-bottom flask. The flask was equipped with a gas joint and a stirrer bearing/shaft/paddle assembly and a 125 ml dropping funnel containing 4.6 g of glycolide. The mixture was evacuated and filled with argon five times to remove residual air and moisture. The reaction apparatus was immersed in a preheated oil bath at 135° C., connected to an argon source with an oil bubbler, and stirred at a moderate speed until all of the solid monomer had melted.

At this time, a volume of stock stannous octoate solution (about 130 mg/ml in toluene) equivalent to 3.6 mg tin (120 ppm stannous octoate or 35 ppm tin) was added to the melt using a 50 μl syringe. The reaction mixture was allowed to stir under a slight argon pressure for approximately 16 hours. At this time the glycolide was melted using a heat gun and added to the polymer melt in the flask. The melt was stirred for an additional 2 hours. The oil bath temperature was then reduced to about 115° C. and the residual monomer was removed under vacuum. The upper parts of the reaction assembly were heated gently with a heat gun to aid in the monomer removal. The total time under vacuum was 2 hours.

The molten prepolymer was suspended in 84 ml of chloroform with stirring and 2.5 equivalents of TEA and 0.5 equivalents of DMAP were added to the stirring reaction mixture using a powder funnel. The reaction mixture was chilled to about 4° C. in a cold bath. A solution of about 1 equivalent of distilled ethyl dichlorophosphate (EOPCl₂) in 27.5 ml of chloroform was prepared in a dropping funnel. The solution in the funnel was added slowly to the reaction mixture over a period of 1 hour. After the addition was complete, the reaction mixture was allowed to stir at low temperature for another 1.75 hours and then the cold bath was removed. The reaction mixture was allowed to warm to room temperature and stirred for 2 to 18 hours. After 2 hours a significant increase in viscosity of the clear solution was observed. The reaction was then quenched with 1 ml of anhydrous methanol and stirred for another five minutes.

Next, 37 g of dry Dowex HCR-S IER and 30 g of dry Dowex M43 were added to the reaction mixture and stirring was continued for another hour to remove residual DMAP and TEA free base and salts. The IERs were removed from the reaction mixture by vacuum filtration through Whatman 54 filter paper. The resin was washed with about one bed volume of dichloromethane and the filtrate was concentrated to approximately 50 ml. The viscous filtrate was poured into 700 ml of petroleum ether to precipitate the polymer and dried under vacuum.

### EXAMPLE 6

### Synthesis of D,L-PL(PG)HOP

All glassware was dried for a minimum of 2 hours at 105° C. and allowed to cool in a desiccator or cooled under a stream of argon gas. A 28.5 g portion of D,L-lactide and 1.5 g of PG (molar ratio, 10:1) were weighed into a 250 ml 3-neck round-bottom flask. The flask was equipped with a gas joint and a stirrer bearing/shaft/paddle assembly. The mixture was evacuated and filled with argon five times to remove residual air and moisture. The reaction apparatus was immersed in a preheated oil bath at 135° C., connected to an argon source with an oil bubbler, and stirred at a moderate speed until all of the solid monomer had melted.

At this time, a volume of stock stannous octoate solution (about 130 mg/ml in toluene) equivalent to 3.6 mg tin (120 ppm stannous octoate or 35 ppm tin) was added to the melt using a 50 μl syringe. The reaction mixture was allowed to stir under a slight argon pressure for approximately 16 hours. The oil bath temperature was then reduced to about 110° C. and the residual monomer was removed under vacuum. The upper parts of the reaction assembly were heated gently with a heat gun to aid in the monomer removal. The total time under vacuum was 2-3 hours.

The molten prepolymer was dissolved in 100 ml of chloroform with stirring and TEA and DMAP were added to the stirring reaction mixture using a powder funnel. The funnel was rinsed with 10 ml of chloroform. The reaction mixture was chilled to about 4° C. in a cold bath. A solution of about 1 equivalent of distilled hexyl dichlorophosphate (HOPCl₂) in 27.5 ml of chloroform was prepared in a dropping funnel. The solution in the funnel was added slowly to the reaction mixture over a period of 1 hour. After the addition was complete, the reaction mixture was allowed to stir at low temperature for another hour and then the cold bath was removed. The reaction mixture was allowed to warm to room temperature and stirred for 2 to 18 hours. After 2 hours a significant increase in viscosity of the clear solution was observed. The reaction was then quenched with 800 μl of anhydrous methanol and stirred for another five minutes.

Next, Dowex MR-3C ion exchange resin (IER) was added to the reaction mixture and stirring was continued for another hour to remove residual DMAP and TEA free base and salts (the Dowex resin had been washed with several bed volumes of methanol and dried under vacuum at ambient temperature for about 18 hours). The resin was removed from the reaction mixture by vacuum filtration through Whatman 54 filter paper. The resin was washed with about one bed volume of dichloromethane and the filtrate was concentrated to approximately 100 ml. The viscous filtrate (now a somewhat cloudy solution) was poured into 1000 ml of hexane to precipitate the polymer. The polymer mass was washed with 2×200 ml of hexane and dried under vacuum. The molecular weight and IV data for the polymers prepared by this process are listed in the table below.

| Sample | Mw (LS), daltons | Mw (CC), daltons | IV, dL/g |
|---|---|---|---|
| 1 | 64,200 | 58,000 | 0.48 |
| 2 | 68,000 | 62,700 | 0.43 |

### EXAMPLE 7

### Synthesis of D,L-PL(PG)EP

All glassware was dried for a minimum of 2 hours at 105° C. and allowed to cool in a desiccator or cooled under a stream of argon gas. A 28.5 g portion of D,L-lactide and 1.5 g of PG (molar ratio, 10:1) were weighed into a 250 ml 3-neck round-bottom flask. The flask was equipped with a gas joint and a stirrer bearing/shaft/paddle assembly. The mixture was evacuated and filled with argon five times to remove residual air and moisture. The reaction apparatus was immersed in a preheated oil bath at 130° C., connected to an argon source with an oil bubbler, and stirred at a moderate speed until all of the solid monomer had melted.

At this time, a volume of stock stannous octoate solution (about 130 mg/ml in toluene) equivalent to 120 ppm stannous octoate or 35 ppm Sn was added to the melt using a syringe. The reaction mixture was allowed to stir under a slight argon pressure for 4 hours. The oil bath temperature was then reduced to about 110° C. and the residual monomer was removed under vacuum. The upper parts of the reaction assembly were heated gently with a heat gun to aid in the monomer removal. The total time under vacuum was 2 hours.

The molten prepolymer was dissolved in 84 ml of chloroform with stirring and 2.5 equivalents of TEA and 0.5 equivalents of DMAP were added to the stirring reaction mixture using a powder funnel. The reaction mixture was chilled to about −5° C. in a cold bath. A solution of about 1 equivalent of distilled ethyl dichlorophosphonate (EPCl₂) in 9 ml of chloroform was prepared in a dropping funnel. The solution in the funnel was added slowly to the reaction mixture over a period of 0.5 hour. After the addition was complete, the viscosity of the solution had increased significantly and the reaction mixture was allowed to stir at low temperature for 1 hour at −5° C. The reaction was then quenched with 1 ml of anhydrous methanol and stirred for another five minutes.

Next, the reaction mixture was transferred to a 0.5 gallon vessel and mixed with 37 g of Dowex DR-2030 IER and 30 g of Dowex-43, and shaken on a mechanical shaker for 2 hour to remove residual DMAP and TEA free base and salts (the IERs had been washed with several bed volumes of methanol and chloroform and dried under vacuum at ambient temperature for about 18 hours). The resin was removed from the reaction mixture by vacuum filtration through Whatman 54 filter paper. The resin was washed with about one bed volume of dichloromethane and the filtrate was concentrated to approximately 50 ml. The viscous filtrate was poured into 200 ml of petroleum ether to precipitate the polymer. The polymer mass was washed with 100 ml of petroleum ether and dried under vacuum. The molecular weight data for the polymers prepared by this process are listed in the table below.

| Sample | Mw (LS), daltons | Mw (CC), Daltons |
|---|---|---|
| 1 | 339,900 | 327,600 |
| 2 | 369,800 | 360,900 |

### EXAMPLE 8

### Synthesis of P(cis- and trans-CHDM/HOP)

All glassware was dried for a minimum of two hours at 105° C. and allowed to cool in a desiccator or cooled under a stream of argon gas. A reaction assembly consisting of a 1 L three-neck round-bottom flask equipped with a gas joint, a stirrer bearing/shaft/paddle and a dropping funnel. A solution of 20.0 g of 1,4-cyclohexane dimethanol (CHDM) was prepared in 75 ml of anhydrous tetrahydrofuran (THF) and transferred to the reaction vessel. The beaker was rinsed with 25 ml of THF and the wash was transferred to the reaction vessel.

Next, 29.0 ml of N-methylmorpholine (NMM) and 1.61 g of DMAP were added to the reaction mixture through a powder funnel. A solution of 28.86 g of hexyl dichlorophosphate (HOPCl₂) in 30 ml of THF was prepared under argon and transferred to the dropping funnel while the reaction mixture was cooled to 4° C. in a cold bath. The solution in the funnel was added to the reaction mixture over a period of one hour. With 5 to 10 minutes after the start of addition, a white precipitate, presumably the hydrochloride salts of NMM and DMAP, began to form. After the addition was complete the funnel was rinsed with 30 ml of THF. The reaction mixture was stirred for 1 hour at 4° C. and then for either 2 or 18 hours at ambient temperature.

At the prescribed time, the precipitate was removed from reaction mixture by vacuum filtration. The filtrate was diluted with 100 ml of dichloromethane, transferred to a half-gallon jar and 86.5 of dried Dowex HCR-S IER and 103.8 g of dried Dowex M43 IER were added to the filtrate. The jar was sealed with a Teflon lined lid and the mixture was agitated on a mechanical shaker for two hours.

At this time, the IERs were removed by vacuum filtration and the filtrate was concentrated to approximately 100 ml under vacuum. The polymer solution was poured in 2 L of hexane and the resulting fluid material that precipitated was isolated and transferred to a Teflon lined glass dish. The polymer was dried under vacuum to yield a sticky, free flowing viscous liquid. The Mw (LS) data for the polymers prepared by this process are listed in the table below.

| Sample | Mw (LS), daltons | Mw (CC), daltons | IV, dL/g |
|---|---|---|---|
| 1 | 4400 | 5500 | 0.14 |
| 2 | 5000 | 6500 | 0.11 |
| 3 | 4000 | 4600 | 0.10 |

### EXAMPLE 9

### Synthesis of P(BHET/EOP)

All glassware was dried for a minimum of two hours at 105° C. and allowed to cool in a desiccator or cooled under a stream of argon gas. A reaction assembly consisting of a 500 ml three-neck round-bottom flask equipped with a gas joint, a stirrer bearing/shaft/paddle and a dropping funnel. First, 30.0 g of bis(hydroxyethyl) terephthalate (BHET) and 28.83 g of DMAP were added to the reaction vessel using a powder funnel and mixed with 81 ml of THF. The solids were dissolved with stirring and gentle heating using a heat gun.

After all solids had dissolved, the reaction mixture was cooled to 4° C. in a cold bath. A solution of 19.2 g of ethyl dichlorophosphate (EOPCI₂) in 24 ml of THF was prepared in a 125 ml addition funnel. The solution in the funnel was added to the solution in the flask over a period of 1 hour. Shortly after the addition had begun, a white precipitate, presumably DMAP hydrochloride, began to precipitate from the reaction mixture. After all of the solution in the funnel had been added, the stirrer shaft/paddle became entrapped in a thick, stiff precipitate and stirring ceased. It appears the polymer that had formed at this time was insoluble in the reaction mixture.

Next, 125 ml of dichloromethane were added and the reaction mixture was swirled by hand until mechanical stirring could be resumed. The reaction mixture was now a homogenous solution containing a white free flowing powder. The reaction mixture was stirred at 4° C. for one hour. The cold bath was removed and the reaction mixture was allowed to warm to ambient temperature and stirred for 16 hours. At this time, the white precipitate was removed from the reaction mixture by vacuum filtration and the filter cake was washed with 100 ml of dichloromethane.

The resulting filtrate was transferred to a half-gallon jar and treated with 156.92 g of undried Dowex HCR-S IER and 160.92 g of undried Dowex M43 IER. The resins were washed with 2 bed volumes of methanol and 2 bed volumes of dichloromethane prior to use. The jar was sealed with a Teflon lined lid and shaken on a mechanical shaker for two hours. The resin was removed by vacuum filtration and the filtrate, ˜600 ml, was concentrated to ˜150 ml. The clear solution was poured into 1.2 L of hexane. The thick oil that precipitated was washed with 400 ml of hexane and transferred to a Teflon lined glass dish, dried under vacuum. The molecular weights were determined by GPC were 2200 for Mw (LS) and 2100 for Mw (CC). The value obtained for IV was 0.10 dL/g.

### EXAMPLE 10

### Synthesis of P(BHET-EOP/TC)

All glassware was dried for a minimum of two hours at 105° C. and allowed to cool in a desiccator or cooled under a stream of argon gas. A reaction assembly consisting of a 500 ml three-neck round-bottom flask equipped with a gas joint, a stirrer bearing/shaft/paddle and a dropping funnel. First, 30.0 g of BHET and 28.83 g of DMAP were added to the reaction vessel using a powder funnel and mixed with 81 ml of THF and 125 ml of dichloromethane.

The solids were dissolved with stirring and gentle heating using a heat gun. After all solids had dissolved, the reaction mixture was cooled to 4° C. in a cold bath. A solution of 19.2 g of EOPCl₂ in 24 ml of THF was prepared in a 125 ml addition funnel. The solution in the funnel was added to the solution in the flask over a period of 1 hour. Shortly after the addition had begun, a white precipitate, presumably DMAP hydrochloride, began to precipitate from the reaction mixture. The reaction mixture was stirred at 4° C. for one hour. Next, a solution of 4.79 g of terephthaloyl chloride (TC) in 18 ml of THF was prepared in the addition funnel and added to the solution in the flask over a 30-minute period. The reaction mixture was stirred for one hour at 4 C.

At this time the cold bath was removed and the reaction was allowed to warm to room temperature and stir for another 20 hours. At this time, the white precipitate was removed from the reaction mixture by vacuum filtration. The resulting filtrate was transferred to a half-gallon jar and treated with 88.5 g of dried Dowex HCR-S IER and 73.8 g of dried Dowex M43 IER. The jar was sealed with a Teflon-lined lid and shaken on a mechanical shaker for two hours. The resin was removed by vacuum filtration and the filtrate was concentrated to ˜100 ml. The clear solution was poured into 2 L of hexane. The thick oil that precipitated was transferred to a Teflon-lined glass dish, dried under vacuum. The molecular weights were determined by GPC were 7200 for Mw (LS) and 4000 for Mw (CC). The value obtained for IV was 0.09 dL/g.

### EXAMPLE 11

### Large-Scale Preparation of D,L-PL(PG)EOP

A 100 g portion of propylene glycol was added to a 3000 ml 3-necked round bottom flask equipped with a gas joint, a stirrer bearing/shaft/paddle assembly, and a Teflon-coated thermocouple. The reaction apparatus was placed in a preheated oil bath at 130° C. and purged with nitrogen for one minute. A 2000 g portion of D,L-lactide was added using a powder addition funnel over a period of 45 minutes. The reaction apparatus was then immersed in the oil so that the oil level was at the bottom of the ground glass joints. The mixture was stirred until all of the solid monomer had melted and the internal temperature had reached approximately 125° C. At this time, a volume of solution of stannous octoate in chloroform equivalent to approximately 400 ppm (117 ppm Sn) was added to the melt using a syringe. The mixture was allowed to stir for approximately 3-16 hours. Then oil bath set point was decreased to approximately 125° C. and any residual unreacted monomer removed using vacuum over approximately 1 hour.

A 2500 ml portion of chloroform was used to dissolve and transfer the prepolymer to a pre-chilled, 20-liter jacketed reactor, which contained 2.5 equivalents (based on propylene glycol) of triethylamine and 0.5 equivalents of DMAP dissolved in 3600 ml of chloroform. The reactor was equipped with a stirrer bearing/shaft/turbine assembly, a gas joint, a tubing adapter, and a Teflon-coated thermocouple. With stirring and chilled recirculation on the jacket, the solution was cooled to below −15° C. A solution of 1 equivalent (based on propylene glycol approximately 215 g) of distilled ethyl dichlorophosphate (EOPCl₂) in 650 ml chloroform was prepared in a 1000 ml 3-necked round bottom flask equipped with a tubing adapter and a gas joint. The EOPCl₂/chloroform solution was added using a piston pump and Teflon tubing over a period of 50 minutes, maintaining the internal temperature at approximately −10° C. Tubing was connected to the gas joints of the flask and reactor to equalize the pressure during the addition. Following the addition, a 50 ml portion of chloroform was added to rinse the flask, feed lines, and pump. The reaction mixture was stirred for 1 hour at low temperature (−8° C. after 1 hour) before the reaction was quenched with 140 ml of anhydrous methanol.

The reactor was then charged with 3 kg of Dowex DR-2030 IER and 3 kg of Dowex M-43 wetted with approximately 6.5 liters of methylene chloride. The polymer/resin mixture was mixed at low temperature for 3-15 hours, after which it was transferred by vacuum to a stainless steel laboratory Nutsche filter. After filtering off the resin, the polymer solution was pulled through the in-line 8 micron cartridge filter into the concentrator (a similar 10-liter jacketed reactor) where the solution was concentrated with the aid of heated recirculating fluid on the jacket. The 20-liter reactor and the resin in Nutsche were washed with 5 liters of methylene chloride, which were transferred to the concentrator after being stirred for 1 hour. An additional 5 liters of methylene chloride were added to the resin in the Nutsche and added to the concentrator when the solution had been reduced to approximately 6 liters.

Concentration of the polymer solution continued until approximately 4-5 liters of a viscous solution remained. A portion of 1500 ml of ethyl acetate was then added to the polymer solution. The mixture was mixed until homogenous and precipitated in approximately 10 liters of petroleum ether. After the precipitation mixture was stirred for approximately 5 minutes, the supernatant liquid was decanted. The polymer was then washed with 5 liters of petroleum ether. After the mixture was stirred for 5 minutes. The liquid was again decanted. The polymer was poured into a Teflon-coated pan and placed in the vacuum oven at NMT 50° C. After drying for 24 hours, the polymer was ground into smaller pieces and dried for additional time in a vacuum oven at ambient temperature.

### EXAMPLE 12

### Encapsulating Paclitaxel into the Subject Polymers

The term “PACLIMER” shall refer to a subject polymer in a microsphere form with the D,L-PL(PG)EOP composition containing paclitaxel at certain loading levels. The D,L-PL(PG)EOP polymer in PACLIMER may be prepared using the method described in Example 1, 2 or 11. If there is no designation after PACLIMER, then paclitaxel is loaded at the 10% level; otherwise, the loading level will be expressly stated or alternatively indicated in parentheses as shown for the following examples: for 30% loading level, “PACLIMER (30%)”; for fifty percent loading, “PACLIMER (50%)”; etc. All microspheres, unless otherwise indicated, were prepared using the solvent dilution method described below.

The four methods listed below may be applied to a variety of drug in polymer loadings:

Method 1—Spray Drying: 10 g of a phosphorous linked polymer, e.g., D,L-PL(PG)EOP, is dissolved in methylene chloride at a concentration of about 10%. After the polymer is completely dissolved, an appropriate amount of paclitaxel powder (e.g., 1.1 g for 10% loading, 4.2 g for 30% loading, 10 g for 50% loading, etc.) is added to the solution and stirred until the powder is completely dissolved. Microspheres are then prepared using a spray-drying technique, e.g., using a Buchi Mini Spray Dryer (Model B-191) at inlet temperature of 35° C., pump rate of 16% (˜10 gm/min) for polymer solution and 800 L/hr for atomizer gas (nitrogen), and aspiration at 50% (˜20 mbar). In most instances, the mean diameter of the resulting microspheres for PACLIMER at various loading levels is less than about 20 microns.

Method II—Solvent Evaporation: Microparticles of the subject compositions will be prepared by solvent evaporation. For example, the subject polymer composition and paclitaxel are dissolved in ethyl acetate, the ethyl acetate solution is then emulsified into a 0.5% polyvinylalcohol (PVA) solution presaturated with ethyl acetate at a stirring rate of 600 rpm, followed by the application of a vacuum (e.g., about 15 inches of Hg) to remove the ethyl acetate. For one exemplary process, the ethyl acetate concentration will be reduced to below 10% within 10 minutes. Microparticles will be washed on an appropriate sieve with deionized water and thereafter lyophilized.

Method III—Solvent Dilution: Microspheres may be prepared by a solvent dilution method using an in-line homogenizer. For example, approximately 50 grams of paclitaxel and 450 grams of subject polymer composition were weighed and dissolved in 1 L of ethyl acetate. The non-solvent phase was pre-saturated with ethyl acetate; ethyl acetate (800 ml) was added to 9 liter of 0.5% PVA and homogenized for 1 minute. The paclitaxel-subject polymer composition solution and the PVA-ethyl acetate solution were pumped simultaneously through an in-ine homogenizer into a container at rates of 1 and 3 liters/min, respectively. The combined solution was gently stirred with an overhead stirrer. Approximately 90 liters of water was added to the container at a rate of 3 L/min. The solution was then gently stirred for 30 minutes. The microsphere suspension was transferred to a filtering/drying unit containing 150 μM scalping and 25 μm product sieves. The resulting microspheres were rinsed with 5 liters of de-ionized water and dried for 3 days under vibration, vacuum and a nitrogen purge. The dried microspheres on the 25 μm sieve were collected into a container.

Method IV—Freeze/pulverize: Microparticles are prepared by evaporating the drug/polymer in solution at 40° C. under a nitrogen purge to obtain viscous mass which is subsequently cooled to −40° C., lyophilized, e.g., for 48 hours, and pulverized to a desired size for the microparticles.

### EXAMPLE 13

### Animal efficacy studies with PACLIMER

### EXAMPLE 13A

### In Vitro Release of Paclitaxel

The PACLIMER microspheres were combined with PEG-1000 at the desired ratio by weight in a 37° C. water bath, mixed to obtain a uniform slurry and then cooled to 4° C. 10 mg aliquots of the PACLIMER/PEG-1000 suspension were pressed into discs for surgical implantation in the rat brain using a pre-cut die. Control implants were made in an analogous fashion but omitting paclitaxel.

Paclitaxel release from the PACLIMER microspheres was quantified by incubating 10 mg of microspheres in 60 mL of phosphate buffered saline (PBS, pH-7.4) at 37° C. Octanol was placed on top of the PBS to continuously extract the released Paclitaxel from the PBS. Paclitaxel concentration in the octanol was assayed by high-performance liquid chromatography (HPLC).

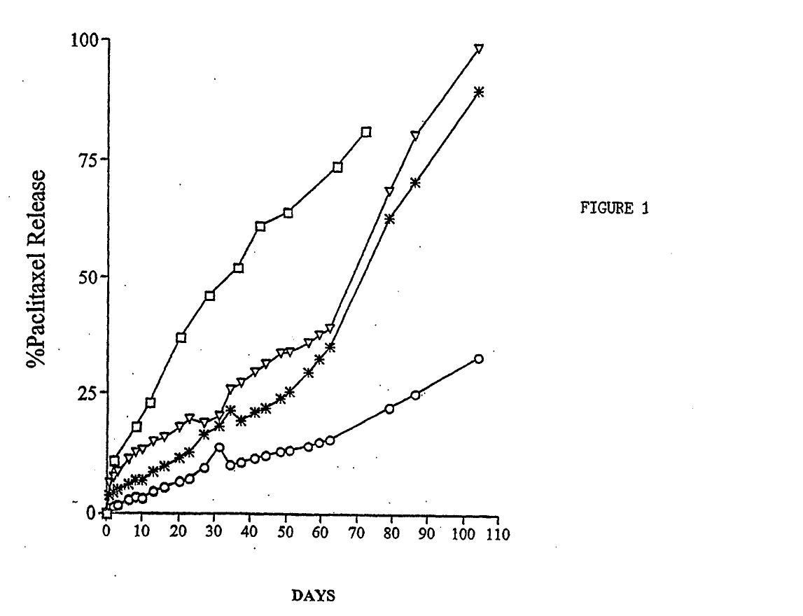

Using high performance liquid chromatography, a near constant rate of paclitaxel release from the PACLIMER microspheres was observed for 90 days in solution (FIG. 1). Because we wished to measure the distribution of paclitaxel from the microspheres in the rat brain, whose limited volume makes injection of a microsphere bead slurry impossible, we also combined the microspheres with varying amounts of polyethylene glycol, M. W. 1000 (PEG-1000). PEG-1000 is solid at room temperature but liquid at 37° C. We were thus able to model an injection by implanting solid PACLIMER:PEG-1000 discs that rapidly liquefy to free PACLIMER microspheres once implanted. Incorporation of PEG-1000 at ratios of 50% or 75% to PACLIMER slightly reduced the initial burst release of paclitaxel compared to free microspheres in vitro, but did not change the slope of the release curve at later time points. Conversely, compressing PACLIMER microspheres into discs without PEG-1000 slowed the release rate of paclitaxel, indicating that the increased surface area of free microspheres is related to the release rate of paclitaxel.

### EXAMPLE 13B

### Biocompatibility and Toxicology

Rat 9 L gliosarcoma cells were grown in Dulbecco's Modified Essential Medium (Gibco-BRL) with 4.5 g/L glucose, supplemented with 10% fetal bovine serum, and penicillin/streptomycin. Tumor allografts were initiated by injecting 1×10⁶ cells into the flanks of Fischer 344 rats. Mature tumors were harvested from anesthetized animals and cut into 2 mm pieces on ice for intracranial implantation.

Male Fischer 344 rats weighing 200-225 p, were obtained from Harlan Sprague-Dawley, Inc. (Indianapolis, Ind.), kept in standard animal facilities with 4 rats/cage, and given free access to food and water. All surgical procedures were performed using intraperitoneal ketamine/xylazine anesthesia. Animals to be necropsied were anesthetized and then perfusion fixed with 4% phosphate buffered paraformaldehyde. Rat brains were removed, embedded in paraffin, sectioned, and stained with hematoxylin and eosin.

Tumor pieces and PACLIMER:PEG-1000 discs were implanted in an analogous fashion. A 3 mm burr hole was made in the skull 5 mm posterior and 3 mm to the right of the bregma. The dura was incised sharply and the underlying cortex was resected with light suction. Hemostasis was obtained with light compression using sterile gauze and the wound was subsequently irrigated. Dissected pieces of 9 L tumor or PACLIMER:PEG-1000 discs were implanted in the resection cavity and the wound was closed with surgical clips (Clay Adams, Parsippany, N.J.). For animals receiving both 9 L tumor implants and PACLIMER:PEG-1000 discs, the tumor piece was implanted on Day 0 as detailed and the wound closed. On Day 5 the animal was reanesthetized, the clips removed and the PACLIMER:PEG-1000 disc placed on top of the tumor implant. The would was then reclosed with surgical clips. Survival studies were analyzed using the non-parametric Mantel-Cox method using SPSS software (Chicago, Ill.).

PACLIMER:PEG-1000 implants were prepared as above with the exception that ³H-labeled paclitaxel was incorporated into the synthetic process. The final specific activity of ³H-labeled paclitaxel in the PACLIMER:PEG-1000 implant was 1.2 pCi/mg. Rats receiving labeled implants were euthanized in groups of four at 7 and 30 days post implant. The brains were removed and snap frozen in heptane over dry ice. Any gross residual PACLIMER:PEG-1000 implant was removed. The frozen brains were divided into implant and contralateral hemispheres and sectioned coronally at 2-mm intervals. Each section was weighed, dissolved in Solvable homogenizing solution, and combined with Atomlight scintillation cocktail (both New England Nuclear Dupont). The samples were counted on a Beckman LS 6500 liquid scintillation counter. To convert cpm/mg tissue to paclitaxel concentration, a section of tissue adjacent to the polymer was minced and extracted with ethanol. The extract was run on a silica thin layer chromatography plate (Sigma, St Louis, Mo.) spotted with cold paclitaxel and developed in an iodine chamber. The percentage of signal migrating with intact paclitaxel was determined by counting sections of the plate in a scintillation counter.

Examination of brains from rats sacrificed by perfusion fixation at 7,14, 30 days and 12-weeks post-implantation revealed a stereotypic acute neutrophil response, followed by a more chronic macrophage response. At more extended time points, giant cells become evident, consistent with foreign body reaction and polymer degradation. The severity of the immune response at each time point was compared to that elicited by poly[1,3-bis(carboxyphenoxy)propane-co-sebacic-acid], or PCPP-SA, which is used clinically as an intracranial implant. In greater detail, H&E sections (100× magnification) of perfusion-fixed rat parietal lobe 7 days post-implantation for the polymer used in PACLIMER and PCPP-SA demonstrated a polymorphonuclear (PMN) infiltrate adjacent to void spaces corresponding to the sites of polymer implantation. The severity of the PMN reaction was more profound and extended a longer distance from the implant site in the PCPP-SA animals than the animals implanted with the polymer used in PACLIMER. H&E sections (40× magnification) of parietal lobe at 30-days post-implantation in the polymer used in PACLIMER and PCPP-SA treated animals, demonstrated a giant-cell reaction adjacent to the implanted polymer. The degree of foreign body reaction was comparable, however, areas of necrosis were present adjacent to all PCPP-SA implants that were absent from the polymer used in PACLIMER implants. In each case the reaction elicited by the polymer used in PACLIMER implants was judged by a blinded neuropathologist (TI) to be comparable to or less severe than that elicited by PCPP-SA. Additionally, rats were observed daily for feeding and grooming behavior and weighed at repeated intervals. No behavioral or systemic differences between implant and control animals were noted.

The addition of paclitaxel to the microspheres of the polymer used in PACLIMER did not produce any additional overt toxicity. No PACLIMER bearing rats demonstrated any evidence of CNS related toxicity either in terms or mortality, behavior, feeding, grooming, or growth. These results are in contrast to results previously reported with paclitaxel released from the PCPP-SA matrix, where significant toxicity was seen in 25% of rats receiving a PCPP-SA:paclitaxel implant. PCPP-SA releases paclitaxel in a biphasic manner where a significant initial burst or paclitaxel release of up to 50% of the initial loading is delivered within 24 hours of implantation, followed by much slower controlled release. The matrix of the polymer used in PACLIMER eliminates this burst phase and more closely approximates true zero order kinetics, minimizing random toxicity associated with the initial burst.

While no overt signs of paclitaxel toxicity were evident, significant histologic changes associated with paclitaxel were evident. Macrophages invading the implant site were often seen in states of arrested mitosis and early apoptosis in animals bearing the PACLIMER implant. Macrophages at the implant site of animals bearing spheres the polymer used in PACLIMER without paclitaxel entirely lacked these histologic changes. Moreover, these changes were striking up to 12-weeks following implantation indicating biologically active paclitaxel levels are present at the implant site for extended periods. At slightly more distant sites, neuronal architecture appeared normal, indicating a relative selectivity of paclitaxel for dividing cells compared to quiescent ones. A background of reactive astrocytosis and a moderate increase in vascularity is apparent at both time points. These changes were also described in animals treated with paclitaxel administered from a PCPP-SA matrix.

### EXAMPLE 13C

### In vivo biodistribution of paclitaxel released from PACLIMER

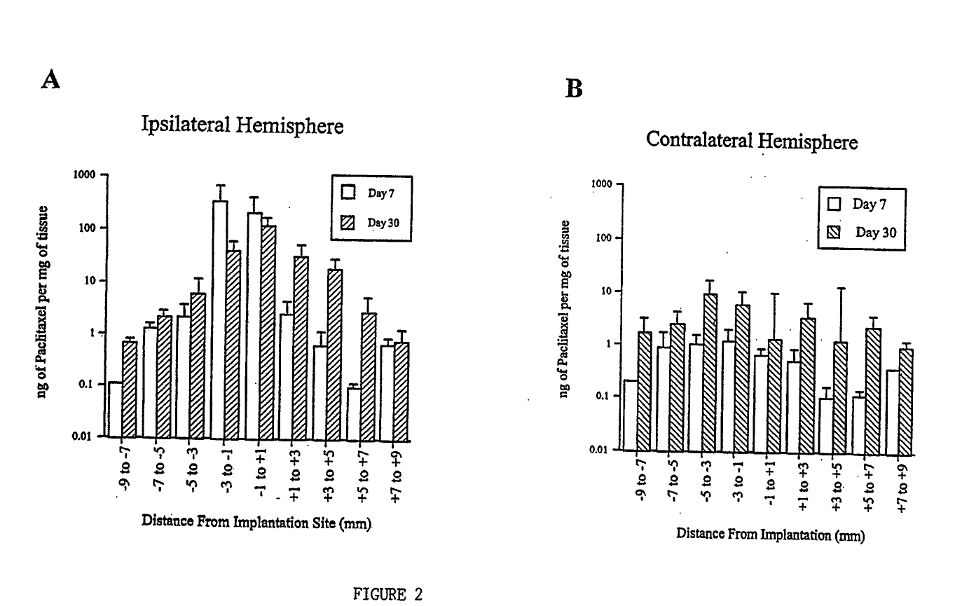

³H-paclitaxel was incorporated into the PACLIMER:PEG-1000 discs in place of unlabeled paclitaxel and implanted intracerebrally in rats via an open craniectomy, as described above. Paclitaxel levels were measured at varying distances from the implant using scintillation counting of tissue homogenates and verified as intact paclitaxel by thin layer chromatography (TLC). At both 7 and 30 days post-implantation paclitaxel levels were detectable throughout the rat brain at levels greater than 1 ng/mg brain tissue (approximately 1 nM), with higher levels detectable at 30 days versus 7 days (FIG. 2). In comparison we have previously reported the LD₉₀ for paclitaxel versus several human glioma cell lines in vitro to be between 5-10 nM. Using this cut-off, therapeutic levels of paclitaxel are achieved 5 to 7-mm from the implant site at 30-days post implantation.

At each time point >90% of raw counts represented intact paclitaxel. The assay could not distinguish between free and tissue-bound paclitaxel. Histologic changes typical of paclitaxel exposure, including arrested mitotic spindles and apoptotic cells, were evident in the brains of rats, containing intracranial PACLIMER implants up to 12-weeks post implantation. This is additional evidence that biologically relevant paclitaxel levels exist in the brain for extended times following implantation.

### EXAMPLE 13D

### Efficacy of PACLIMER versus a CNS neoplasm

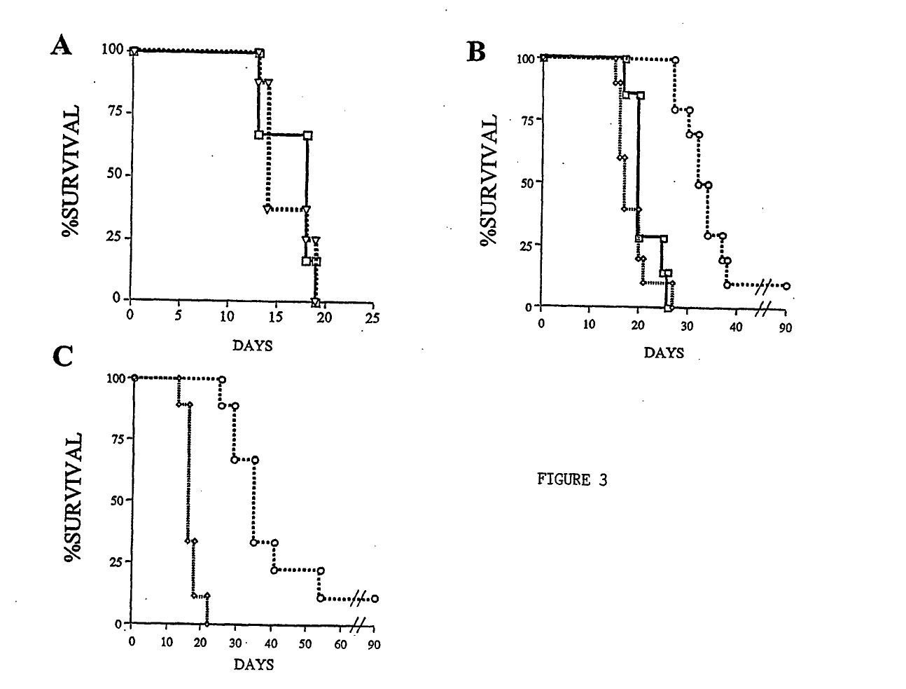

Rats bearing established 9 L glial tumors were prepared and treated with PACLIMER implants as described above. The intracranial 9L glioma model was selected for study because it previously predicted the eventual success of PCPP-SA polymers loaded with BCNU (Gliadel) in Phase III clinical trials. PACLIMER doubled the median survival of rats bearing tumors (35 days versus 16 days, n=10 animals/group, p<0.0001, Kaplan-Meier Method) versus control animals (FIG. 3). All of the animals died with large tumors, verified at necropsy. Empty microspheres had no effect on survival compared to animals receiving no treatment, indicating survival differences were due to paclitaxel release from the matrix. Additionally, an equivalent dose of PACLIMER delivered systemically had no effect on survival indicating that local administration of the PACLIMER may be preferable for efficacy in this system. Each experiment also produced rats that were long-term survivors who had no tumor evident at the time, of sacrifice 90-days post implantation.

In a previous report examining paclitaxel delivery via PCPP-SA implants in rodent models of malignant brain tumors, it was demonstrated that while the implant was efficacious against brain tumors, the matrix released drug in a biphasic pattern with an initial burst of drug followed by a much slower release of drug over months. The burst of paclitaxel resulted in sporadic toxicity among animals implanted with the PCPP-SA:paclitaxel matrix and made the preparation suboptimal for clinical applications.

In contrast, PACLIMER microspheres were able to release paclitaxel in vitro at a constant rate for the entire period of drug release. Correspondingly the sporadic toxicity associated with the unpredictable burst phase of drug release disappeared. At the same time, the immune reaction elicited by the polymer backbone of the polymer used in PACLIMER was comparable or less pronounced than that caused by PCPP-SA, indicating that the polymer used in PACLIMER should be biocompatible in the CNS. Minimizing host-reaction to an intracranial implant is typically helpful in preventing long-term clinical sequelae from the implant such as gliosis, seizures, or neurologic deficit. None of the rats receiving implants of the polymer used in PACLIMER experienced any of these complications.

In addition to affording controlled release of drug, an optimal matrix will be able to protect incorporated drug from degradation prior to release. At 30-days post implant we determined that greater than 90% of drug within the brain corresponded to intact paclitaxel. Furthermore, histologic changes typical of acute paclitaxel exposure were evident in the brains of rats receiving PACLIMER implants up to 12-weeks post implantation consistent with ongoing exposure of the brain to active drug.

### EXAMPLE 13E

### PACLIMER and Radiation Treatment

PACLIMER microspheres were combined with polyethylene glycol 1000 (PEG-1000) at a 1:1 ratio by weight and after thorough mixing, 10 mg aliquots of the PACLIMER/PEG-1000 suspension were pressed into 4×l mm discs. To make the control implants, an analogous procedure was followed while omitting paclitaxel.

Sixty-five female Fischer 344 rats weighing 150-175 g were obtained and housed in standard animal facilities. The animals were divided into five experimental groups (n=13 each): 1) control; 2) PACLIMER-only; 3) radiation (XRT)-only; 4) PACLIMER followed by XRT (P/X); 5) XRT followed by PACLIMER (X/P). On Day 0, all animals underwent direct surgical implantation of a 1-mm3 fragment obtained from a solid 9 L gliosarcoma that had been propagated in a rat flank.

On Day 5, the control group underwent implantation of blank D,L-PL(PG)EOP/PEG-1000 discs. Two groups—PACLIMER-only and P/X—underwent surgical implantation of PACLIMER discs, while the remaining animals—XRT-only and X/P-were irradiated with a single dose of 20 Gy collimated beam centered over the tumor implantation burr hole. All animals undergoing radiotherapy were shielded with a square primary collimator (7×7 cm) and a circular secondary collimator (1 cm diameter).

On Day 10, the order of treatment for the P/X and X/P groups was reversed such that the animals in the P/X group underwent radiotherapy with a single 20 Gy dose as described above, and the X/P animals underwent surgical implantation of PACLIMER in the above-mentioned fashion. The animals were observed for signs of neurological or systemic toxicity, and their survival was recorded.

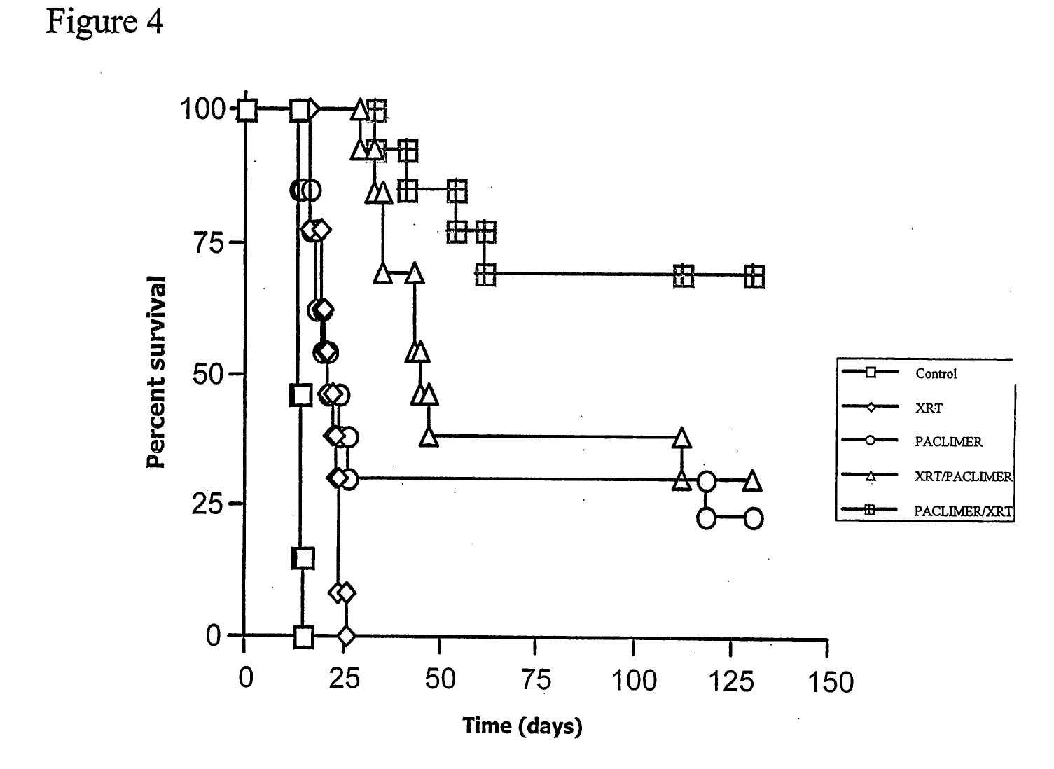

Results from this experiment are shown in FIG. 4. The results are summarized in the table below:

| | No. | Median | % Increase | No. of | | |
|---|---|---|---|---|---|---|
| Treatment | of | survival | in life | long term | | |
| regimen | rats | (days) | span | survivors | P value¹ | P value² |
| Control | 13 | 13 | | 0 | | <0.001 |
| XRT | 13 | 21 | 62 | 0 | <0.001 | <0.001 |
| PACLIMER | 13 | 21 | 62 | 4 | <0.001 | <0.001 |
| XRT/PACLIMER | 13 | 45 | 247 | 5 | <0.001 | 0.004 |
| PACLIMER/XRT | 13 | 130 | 900 | 9 | <0.001 | 0.028 |

¹Kaplan-Meier survival benefit as compared to control treatment group
²Kaplan-Meier survival benefit as compared to PACLIMER/XRT group

As shown, treatment with electromagnetic radiation in combination with PACLIMER (10%) improved survival rates, and treatment with electromagnetic radiation after administration of PACLIMER (10%) resulted in significantly greater survival rates than treatment with electromagnetic radiation before administration of PACLIMER (10%). This result indicates that paclitaxel when released from PACLIMER (10%) is acting as a radiosensitizer as a well as a chemotherapeutic agent. EQUIVALENTS

The present invention provides among other things, compounds, compositions, polymers, and methods. While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

All publications and patents mentioned herein, including those items listed below, are hereby incorporated by reference in their entirety as if each individual publication or patent was specifically and individually indicated to be incorporated by reference. In case of conflict, the present application, including any definitions herein, will control. To the extent that any U.S. Provisional Patent Applications to which this patent application claims priority incorporate by reference another U.S. Provisional Patent Application, such other U.S. Provisional Patent Application is not incorporated by reference herein unless this patent application expressly incorporates by reference, or claims priority to, such other U.S. Provisional Patent Application. Also incorporated by reference are the following:

Patents and patent applications U.S. Pat. Nos. 4,638,045, 5,219,564, 5,099,060, 6,040,330, 6,017,935, 6,002,023, 5,990,325, 5,981,564, 5,977,164, 5,977,163, 5,972,992, 5,922,754, 5,919,815, 5,908,835, 5,912,263, 5,902,822, 5,877,205, 5,854,278, 5,840,929, 5,821,363, 5,817,840, 5,808,888, 5,795,909, 5,780,653, 5,773,464, 5,773,461, 5,767,297, 5,767,296, 5,760,072, 5,756,776, 5,750,691, 5,739,359, 5,728,687, 5,719,177, 5,693,666, 5,688,977, 5,686,623, 5,670,536, 5,614,645, 5,608,087, 5,597,931, 5,908,835, 6,005,120, 5,424,073, and 5,547,981.

Publications and other references
Betz et al. (1994) Chapter 32 in Basic Neurochemistry (5th Edition, Eds Siegel, Albers, Agranoff, Molinoff), pp 681-701.
Brem et al., (1995) Lancet, 345: 1008-12.
Cahan et al. (1994) Cancer Chemother Pharmacol. 33: 44
Chamberlain et al. (1997) J Clin Oncol. 15: 3427-3432
Chamberlain et al. (1999) J Neurooncol. 43: 71-8
Choueka et al., (1996) J. Biomed. Materials Res., 31:35-41)
Dorduno et al. (1995) Cancer Chemother Pharmacol. 36: 279-82
Ertel et al., (1995) J. Biomedical Materials Res. 29:1337-1348
Fountzilas et al. (1999) J Neurooncol. 45: 159-65
Glantz et al., (1995) Sem Oncology. 5, Suppl 12: 26-32
Glantz et al. (1995) J Natl Cancer Inst. 87: 1077-91, 1995.
Gupta et al. (1997) Int J Radiation Oncology Biol. Phys. 37:885-95
Hong et al. (1999) Carcinogenesis. 20: 1161-8
Langer et al., (1983) Rev. Macro. Chem. Phys. C23(1):61
Leong et al. (1986) Biomaterials. 7:364
Prados et al., (1996) J Clin Oncol. 14: 2316-21.
Rosenthal et al., (2000) J Neurooncol. 47: 59.
Sato et al., (1996) Bio. Pharm. Bull. 19:1596-601
Suh et al. (1998) J Biomed Mater Res. 42: 331-8
Walter et al., (1995) Intratumoral Chemotherapy. Neurosurgery 37: 112845
Walter et al. (1994) Cancer Res. 54: 220712
Wehbe et al. (1998) J Neurooncol. 39: 245-51

1. A method for treating a central nervous system neoplasm of a patient, comprising: instilling into an anatomic area of a patient affected by a central nervous system neoplasm a therapeutically effective amount of a composition comprising a biocompatible polymer and an antineoplastic agent, wherein said polymer comprises phosphorous-based linkages.

2. The method of claim 1, wherein said polymer is biodegradable.

3. The method of claim 1, wherein said instillation does not cause a deleterious amount of inflammation in the central nervous system of said patient.

4. The method of claim 1, wherein said antineoplastic agent is an antineoplastic taxane.

5. The method of claim 4, wherein said antineoplastic taxane is paclitaxel.

6. The method of claim 1, wherein said polymer comprises one or more monomeric units represented by the following formula V: 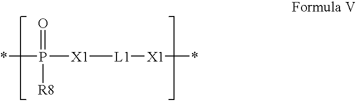
wherein, independently for each occurrence of said monomeric unit:
X1, each independently, represents —O— or —N(R7)—;
R7 represents —H, aryl, alkenyl or alkyl;
L1 represents any chemical moiety that does not materially interfere with the biocompatibility of said polymer;
R8 represents —H, alkyl, —O-alkyl, —O-cycloalkyl, aryl, —O-aryl, heterocycle, —O-heterocycle, or —N(R9)R10;
R9 and R10, each independently, represent a hydrogen, an alkyl, an alkenyl, —(CH₂)ₘ—R11, or R9 and R10, taken together with the N atom to which they are attached complete a heterocycle having from 4 to about 8 atoms in the ring structure;
m represents an integer in the range of 0-10; and
R11 represents —H, alkyl, aryl, cycloalkyl, cycloalkenyl, heterocycle or polycycle.

7. The method of claim 1, wherein said polymer comprises one or more monomeric units represented by the following formula VI: 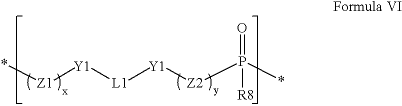
wherein Z1 and Z2, respectively, for each independent occurrence is: 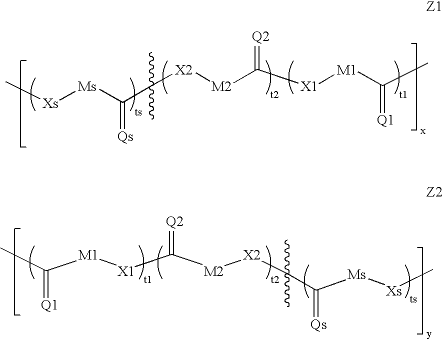
wherein, independently for each occurrence of said monomeric unit:
Q1, Q2 . . . Qs, each independently, represent —O— or —N(R7);
X1, X2 . . . Xs, each independently, represent —O— or —N(R7);
R7 represents —H, aryl, alkenyl or alkyl;
the sum of t1, t2 . . . ts is an integer and equal to at least one or more;
Y1 represents —O—, —S— or —N(R7)—;
x and y are each independently integers from 1 to about 1000 or more;
L1 represents any chemical moiety that does not materially interfere with the biocompatibility of said polymer;
M1, M2 . . . Ms each independently, represents any chemical moiety that does not materially interfere with the biocompatibility of said polymer;
R8 represents —H, alkyl, —O-alkyl, —O-cycloalkyl, aryl, —O-aryl, heterocycle, —O-heterocycle, or —N(R9)R10;
R9 and R10, each independently, represent a hydrogen, an alkyl, an alkenyl, —(CH₂)ₘ—R11, or R9 and R10, taken together with the N atom to which they are attached complete a heterocycle having from 4 to about 8 atoms in the ring structure;
m represents an integer in the range of 0-10; and
R11 represents —H, alkyl, aryl, cycloalkyl, cycloalkenyl, heterocycle or polycycle.

8. The method of claim 1, wherein said polymer comprises one or more monomeric units represented by the following formula VII: 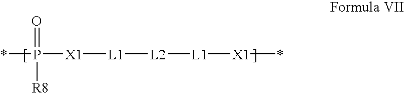
wherein, independently for each occurrence of said monomeric unit:
X1, each independently, represents —O— or —N(R7)—;
R7 represents —H, aryl, alkenyl or alkyl;
L1 represents any chemical moiety that does not materially interfere with the biocompatibility of said polymer;
R8 represents —H, alkyl, —O-alkyl, —O-cycloalkyl, aryl, —O-aryl, heterocycle, —O-heterocycle, or —N(R9)R10;
R9 and R10, each independently, represent a hydrogen, an alkyl, an alkenyl, —(CH₂)ₘ—R11, or R9 and R10, taken together with the N atom to which they are attached complete a heterocycle having from 4 to about 8 atoms in the ring structure;
m represents an integer in the range of 0-10; and
R11 represents —H, alkyl, aryl, cycloalkyl, cycloalkenyl, heterocycle or polycycle; and
L2 represents a divalent, branched or straight chain aliphatic group, a divalent cycloaliphatic group, a phenylene group, or a group of the formula:

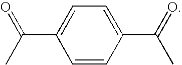

9. The method of claim 1, wherein said polymer comprises one or more monomeric units represented by the following formula VIII: 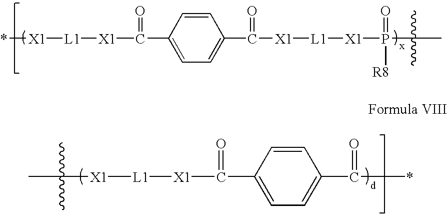
wherein, independently for each occurrence of said monomeric unit:
X1, each independently, represents —O— or —N(R7)—;
R7 represents —H, aryl, alkenyl or alkyl;
L1 represents any chemical moiety that does not materially interfere with the biocompatibility of said polymer;
R8 represents —H, alkyl, —O-alkyl, —O-cycloalkyl, aryl, —O-aryl, heterocycle, —O-heterocycle, or —N(R9)R10;
R9 and R10, each independently, represent a hydrogen, an alkyl, an alkenyl, —(CH₂)ₘ—R11, or R9 and R10, taken together with the N atom to which they are attached complete a heterocycle having from 4 to about 8 atoms in the ring structure;
m represents an integer in the range of 0-10;
R11 represents —H, alkyl, aryl, cycloalkyl, cycloalkenyl, heterocycle or polycycle; and
d is equal to one or more and x is equal to or greater than one.

10. The method of claim 1, wherein said composition provides extended release of said antineoplastic agent into said anatomic area.

11. The method of claim 10, wherein, for a period of at least seven days, the rate of release of said antineoplastic agent is approximately constant.

12. The method of claim 1, wherein said composition releases a therapeutically effective amount of said antineoplastic agent over at least about thirty days after said instillation.

13. The method of claim 1, wherein said anatomic area is on the brain side of the blood brain barrier.

14. The method of claim 1, wherein said composition is at least about 10 percent more effective in treating said central nervous system neoplasm than administration of said antineoplastic agent formulated in a pharmaceutically acceptable carrier without said polymer.

15. The method of claim 1, wherein said method increases the median survival rate from said central nervous system neoplasm by at least about 10 percent as compared with the median survival rate obtained by administration of the same effective dosage of said antineoplastic agent without said polymer.

16. The method of claim 15, wherein said antineoplastic agent is paclitaxel and said antineoplastic agent without said polymer is formulated in 50 percent CREMOPHOR EL and 50 percent dehydrated alcohol.

17. The method of claim 1, wherein said composition increases the median survival rate for a three year period from said central nervous system neoplasm by at least about 50 percent as compared with the median survival rate obtained by administration of a composition comprising the same effective dosage of said antineoplastic agent formulated in a pharmaceutically acceptable carrier.

18. The method of claim 1, wherein said composition reduces the number of hypersensitivity reactions obtained upon administration of said composition by at least about 10 percent as compared with the number of hypersensitivity reactions obtained by administration of a composition comprising the same effective dosage of said antineoplastic agent formulated in a pharmaceutically acceptable carrier and without premedication.

19. The method of claim 1, further comprising treating said patient with electromagnetic radiation.

20. The method of claim 19, wherein said treatment with electromagnetic radiation occurs only before said instillation of said composition.

21. The method of claim 19, wherein said treatment with electromagnetic radiation occurs only after said instillation of said composition.

22. The method of claim 19, wherein said treatment with electromagnetic radiation occurs before and after said instillation of said composition.

23. A composition, comprising: a biocompatible polymer and a therapeutically effective amount of an antineoplastic agent, wherein said composition is suitable for administration to a patient, said composition is in at least partial contact with an anatomic area affected with a central nervous system neoplasm, and said polymer comprises phosphorous-based linkages.

24. (canceled)
